(19) United States
(12) Patent Application Publication
(10) Pub. No.: US 2006/0121590 A1
(43) Pub. Date: Jun. 8, 2006

(54) Electroionic cellular agitation apparatus for influencing the metabolic cellular functions and method therefore

(76) Inventor: Rodolfo C. Speerli, Buenos Aires (AR)

Correspondence Address: Jesus Sanchelima, Esq.;Sanchelima and Associates, P.A., 235 S.W. Le Jeune Rd., Miami, FL, 33134, US

(21) Appl. No.: 11/005,317

(22) Filed: Dec. 7, 2004

(51) Int. Cl. C12N 13/00

(52) U.S. Cl. 435/173.1

(57) ABSTRACT

An electroionic cellular agitation apparatus for the biophysical stimulation of the cellular metabolism of live systems uses a generator emitting high-voltage pulses at time intervals typically comprised between 1 ms and 100 ms, corresponding to a pulse-frequency pf between 1,000 and 10 pulses/second, respectively, and which by means of a capacitive application device generates an electrical flow penetrating the soft and osseous tissues and originating short duration electrical impulsive forces on the ions situated within and outside the cells thus causing an ionic agitation influencing the metabolic characteristics to increase the development of bacterial and fungal cultures, soft and osseous live tissues, and also for the treatment of a variety of ailments, thus attaining an anticipated healing and the reduction of rehabilitation times. The range for the voltage and pulse frequency values are selected such that the RMS value of the electrical current density has a power level that does not cause any significant heating on said tissues.

### FIELD OF INVENTION

The present invention relates to an electrical apparatus causing the agitation of ions within the cells and the intercellular fluids for influencing the metabolic characteristics of live systems by accelerating the development, healing and cicatrizing of live tissues, both osseous and soft of animal and vegetal origin, unicellular organisms included, based on the application of high-voltage low-frequency pulses transmitted to the live system in a capacitive way.

### PRIOR ART

It is well known that the effect of magnetic and electric fields on live beings was observed at the onset of the XX Century when an increase of vegetation was observed beneath high-tension overhead power lines. It was then that the Russian physician Danilewsky started an exhaustive research on the biological effects of the Hertzian waves on the cells and nervous system; his work was reassumed and extended by the Frenchman D'arsonval.

In 1940 another Russian, Lakoski, treated some malignant tumors with high-frequency waves, and in the 50's the Japanese Fukada and Yesuda and the Americans Basset and Pilla diversified their research to understand the behavior of said electromagnetic waves which increase the electrical potential of the cells, improve the enzyme kinetics, and reduce the reproduction time for soft and osseous tissues, therewith achieving a beneficial antiedemateous, antiphlogystic and antialgyc action.

There are nowadays several equipments useful in physiotherapeutics based on electromagnetic waves as short waves, microwaves and magnetotherapeutics. The former two are based on low-voltage very high-frequency waves applied by means of two isolated electrodes forming a capacitor. Said capacitor generates between its plates an electromagnetic field, also of high frequency, and therefore generates heat in the tissues and metallic implants. This kind of heating is technically known as “diathermia”. In many cases it is counterproductive and therefore unwanted.

In recent times it has been recognized that the therapy achieved by the application of a radiofrequency field is not characterized only by the heating of the tissues due to the field effect. A review on this subject can be found in: “Healing by Electromagnetism—Fact or Fiction”. New Scientist Apr. 22, 1976.

On the contrary, magnetotherapeutics, because of being based on variable magnetic fields with a low frequency of 50-60 Herz, does no cause heat, its effects being attributed to the action of the magnetic field on the ions in the tissues.

A combination of both alternatives are the electromagnetic devices using magnetic fields the intensity of which is lower than the magnetotherapeutic devices, but being of the high-frequency type (their frequency being in the range of radiofrequencies). In these combined devices heat is reduced by using low intensity fields, intermittent emissions or by modulating the radiofrequency carrier wave with a low-frequency modulation wave. Another variant of electromagnetotherapeutic devices are those implementing very low intensity electric fields (their intensity being much lower than short wave and microwaves) and high frequencies which are applied capacitively or by means of an antenna thus generating a high-frequency electromagnetic field. These devices also reduce the tissular heating through intermittent emission or by modulating the radiofrequency carrier wave amplitude with a low-frequency modulation wave.

In more recent times it has been widely recognized that the biological stimulation of the calcium and sodium-potassium pumps is caused by the electrical field, same being applied either directly or being induced by the variation of the magnetic field due to the Maxwel/Lenz Law. A review on this subject can be found in “Calcium signaling in lymphocytes and ELF fields” Federation of European Biochemical Societies, volume 301, number 1.5359, R. P. Liburdy-1992.

This same publication concludes that the action of the 60 Hz sinusoidal electric field interactuates with the plasmatic cellular membrane and with the calcium channels situated on same in contrast to the internal cellular structures not involved in said interaction. The cellular membrane having virtually an isolating feature (lipidyc bilayer membrane) it ensues that the field from a sinusoidal wave as well all the movements of the ions (electric current) show up mostly in the intercellular fluids and enter the cell proper in small amounts only.

Regarding present invention, the electroionic cellular apparatus differentiates itself from the prior ones by following features:

It does not use sinusoidal or quasi-sinusoidal waves, square radiofrequency waves, nor low-frequency waves.

It does not use magnetic fields.

It does not cause a significant tissular heating.

It does use an activation signal consisting of high-voltage very low duration pulses, which allows the field to perform both in the intercellular fluids as well within the cell.

It allows selecting the electrical polarity of the pulses applied.

The utilization of high-voltage low-duration pulses separated at time intervals great enough allows to mobilize the ions within the cell, because when there is no pulse, the ions are virtually in electrical balance. Once the pulse has begun, and due to the effect from the electrical field, all the ions mobilize both within and outside the cell. The movement of the ions is directed to cause the cancellation of the factor originating it (Law of Lenz), i.e., they form a field equal and opposed to the external one. The exterior pulse being of short duration, when same is cancelled the ions within the cell and within the intercellular fluid attain their electrical equilibrium (the internal polarization vanishes). At this moment the next pulse comes up and mobilizes again the ions in the same manner, this process repeating itself as long the pulse emission is going on. This causes the ionic agitation within the cells, increases the enzymatic efficiency and therefore the rate of the metabolic cellular reactions without significatively increasing the temperature, as there is no diathermic effect.

Therefore present invention further to interactuate with the plasmatic membrane, also interactuates with the cell's internal structure, both effects adding themselves and increasing the biophysical stimulation.

The electroionic cellular agitator may be applied to all kind of cellular cultures, both under wet and dry conditions. It can also be used as a therapy system in human beings and higher animals, its minimal treatment intervals being 10 minutes, twice a week. The frequency of sessions depends of the nature of the injury; typically 6 to 20 sessions are required. The system not presenting collateral effects, it is possible to implement up to two or more sessions a day.

### SUMMARY

Accordingly the primary object of the present invention is to provide an apparatus generating high-voltage pulses for influencing a live system's metabolism, said pulses being applied continuously or intermittently for short periods of time (e.g., during 10 to 20 minutes) said pulses being transmitted in a capacitive way to the subject to be treated, thus optimizing the penetration of the electric field both outside as well inside the cells and without causing any substantial heating to the system being treated.

In general terms and within the scope of present invention an electrical apparatus is provided causing an electroionic agitation within the cells in such a manner to influence the metabolic characteristics of live systems, comprising means, for generating a biological activation signal because of consisting of high-voltage pulses with a predetermined characteristic regarding their waveform and pulsewidth, as well the time interval between pulses, and the means for the rhythmical activation for applying said pulses with said characteristics repetitively for time intervals during a total time of treatment.

More specifically, and according to the present invention, a device is provided for a capacitive application to influence the metabolic characteristics of live systems consisting e.g of bacteria, fungi and cell cultures, in order to accelerate the development of same and increasing the production rate of the products obtained from the cellular metabolism of said cultures. The capacitive application device comprises the means for receiving the high-voltage pulses and generating an electrical flux which penetrates the live system and interactuates with the inner and outer parts of the cells thus originating an ionic agitation that alters and enhances the cellular metabolism but without producing any significant heating to the system, as the RMS value of the capacitively-induced electrical current density has a power level such that the heat produced does not cause a significant increase of said system's temperature.

Another aspect of present invention is oriented to the providing of a capacitive application device for biomedical therapeutic applications. The capacitive application device comprises the means for receiving the high-voltage pulses and generating and focusing an electrical flux penetrating the tissues in the area to be treated, and interactuates uniformly in the interior of the cells, originating a ionic agitation which alters and increases the cellular metabolism, without causing any significative heating in the tissues, as the RMS value of the electrical current density, capacitively induced, has a energy level such that the heat caused does not originate a significative increase of the temperature within the tissues treated and nowhere in the patients body.

The apparatus according to present invention preferably comprises:

means for generating al least an activation signal formed by high-voltage short duration pulses, periodically separated at regular time intervals;

means for a rhythmical activation for an intermittent operation, said means defining said periodical intervals for the treatment period;

means for defining the emission and pause duration times of said treatment cycle and the whole duration of said treatment as well:

means for manually defining the frequency value of the high-voltage pulses, optionally including:

means for the periodical and automatic sequential variation of said frequency;

means for defining the electrical polarity of the high-voltage pulses in a manual way; and

means for automatically defining said polarity by means of a program;

and means for defining the peak tension value of the high-voltage pulses, and:

means for detecting the correct operation of the apparatus;

means for displaying the indication of the values defined with the means above-mentioned and through with the apparatus is being operated.

The apparatus according to present invention may also include:

means for defining the frequency value of the low-frequency wave in order to modulate the amplitude of the high-voltage pulses, and:

the display means for showing the indication of said frequency for which said apparatus is being operated;

means for simultaneously defining the functioning of the high-voltage generators; and

means for defining the in-phase or out-of-phase functioning of the latter, and:

means for displaying the indication of the simultaneous functioning of said generators, for which the apparatus is being operated.

Another object of the present invention is the providing of an activation signal for optimizing the penetration of the electrical flux within the biological tissues including within the cells proper. Said activation signal is applied to at least a capacitive application device. Said activation signal comprises a sequence of high-voltage pulses their amplitude being modulated by a low-frequency treatment wave of between approximately 5 and 200 Hz, wherein said modulation wave further determines the electrical polarity of the high-voltage pulses. Every high-voltage pulse shows a strongly damped sinusoidal waveform wherein the first peak the value of which is above the remaining ones determines the pulse polarity, the second polarity pulse opposed to the first one may attain at most a 60% of the value of the first one, the third peak of equal polarity as the first one may attain at most 25% of the value of same, thereafter the polarity inverts again thus initiating the final section with a land form and with an tension value below 12% of the first pulse value and with a duration time superior to 1,2 times the duration of the damped pulsation, till the onset of the next pulse. In said latest section the voltage value and the slow variation do not have any biological effect; this section is the reset time during which the ions within and outside the cells attain their electrical equilibrium, i.e. they depolarize.

More particularly and according to present invention the damped sinusoidal wave forming every pulse has a wave frequency typically no above approximately 3,000 Hz. The maximum maximorum value of the first peak value is not less than approximately 3,000 volt measured between the generator output and ground. This activation signal upon being applied to the capacitive application device sets up between said device and the body of the system being treated a potential gradient with the same waveform as said signal and said electrical potential gradient pulses attain maximum maximorum peak values typically not below approximately 300 volt/mm.

In conformity with present invention it can be appreciated that an apparatus producing an activation signal like the one described and applying same by means of a capacitive application device generating a potential gradient like above described, provides satisfactory results by influencing cellular metabolism either through increasing the growth of biological systems or for biomedical applications.

### BRIEF DESCRIPTION OF THE DRAWINGS

The features, nature, and advantages of the present invention will become more apparent from the attached drawings wherein:

FIG. 1 is basic schematic block diagram of an electroionic cellular agitator apparatus according to the present invention;

FIG. 2 is a schematic block diagram of a preferred embodiment of the present invention of one high-voltage pulse generator;

FIG. 3a is the first part of the schematic block diagram of the apparatus shown in FIG. 2;

FIG. 3b is the second part of the schematic circuitry of the apparatus shown in FIG. 2;

FIG. 4 is a schematic block diagram of a second embodiment of the invention with frequency modulation of the electrical mains;

FIG. 5 is a schematic block diagram of another embodiment of the invention with two high-voltage generators;

FIGS. 6 a-b show the wave-like diagrams of the high pulse activation signal for two alternative operation modes corresponding to the diagrams in FIG. 1 to FIG. 5;

FIG. 7 is a wave-like diagram showing output waveforms in several parts of the diagrams in FIG. 1 to FIG. 5;

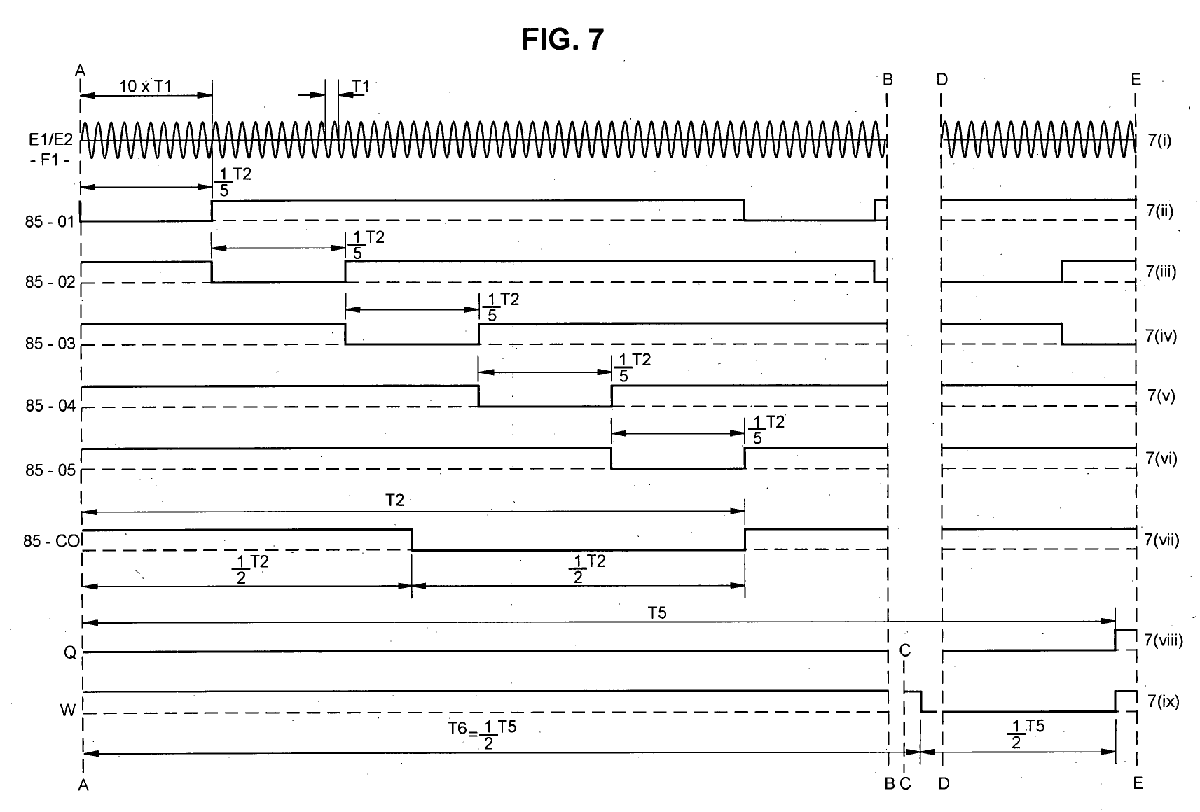

FIG. 8 a-d are a detailed view and cutouts of the connection system between the high pulse generator and the capacitive application device.

FIG. 9 a-b are a view and cutouts of a capacitive application device for biomedical use in human beings and superior animals; and

FIG. 10 a-c are detailed views of a capacitive application device for treating live systems in culture test tubes.

### DETAILED DESCRIPTION OF PREFERRED EMBODIMENTS

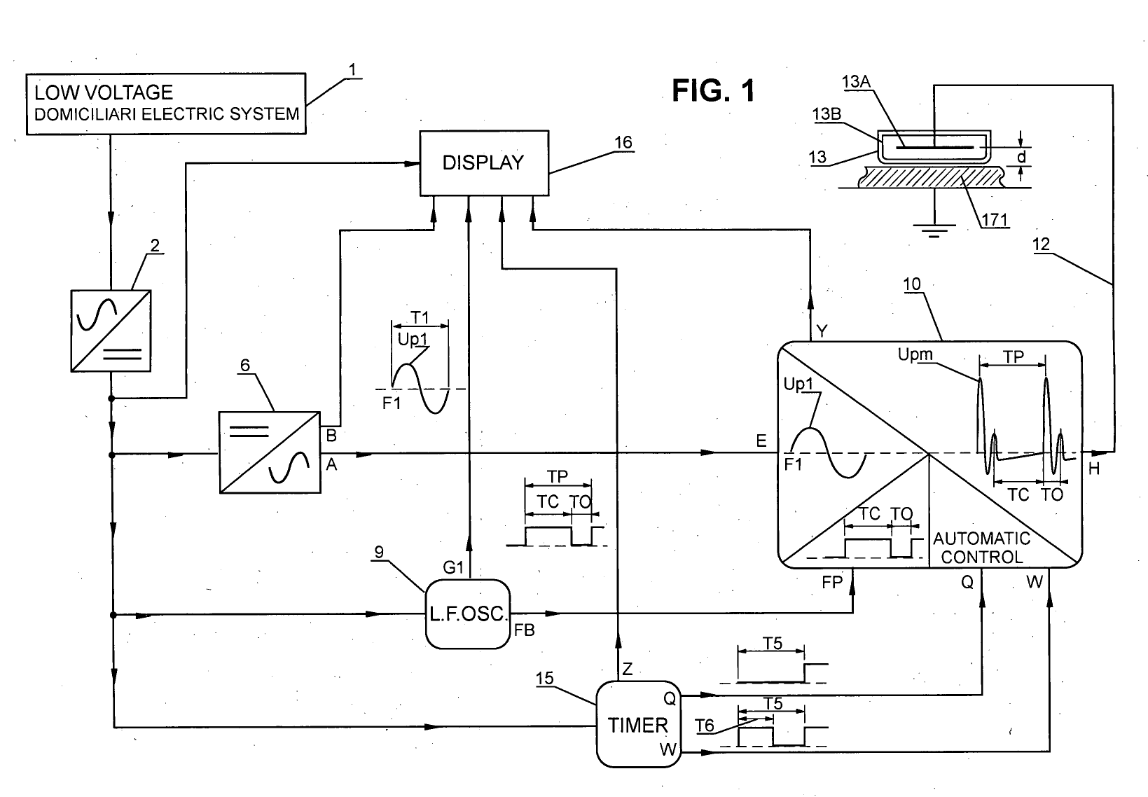

Referring to FIG. 1, the electroionic cellular agitator apparatus comprises basically a power source 2 connected to the mains 1, said power source 2 being a converter transforming the alternate voltage of the power input into a direct (continuous) output power, and may include an electric battery.

The output of power source 2 connects and feeds the inputs of electric ondulator 6, electric oscillator 9, timer 15 and display 16.

Ondulator 6 converts the DC voltage into quasi-sinusoidal alternate current voltage of frequency F1 variable from 5 Hz to 200 Hz and with a peak value Up1 which through its output A connects to and feeds the CCA (convertor-amplifier) circuit 10 through input E of same. Ondulator output B forwards the information on its functioning frequency to display 16.

Oscillator circuit 9 is a digital square wave generator with a variable frequency from 10 to 1,000 Hz, the time interval between pulses being denoted as TP; for logical state 1 (one) the mark time is TC and for logical time 0 (zero) space-time is TO, this making TP=TC+TO.

This pulsation frequency of oscillator 9 through its output FB connects with input FP of CCA 10. Output G1 of oscillator 9 communicates the functioning frequency information to display 16.

The low voltage frequency F1 wave is processed by CCA 10 which amplifies the frequency and voltage transforming it into a strongly damped high-voltage sinusoidal pulsation emitted at the onset of time intervals TO (space time). Further, the pulse voltage value will be amplitude-modulated by same F1 frequency wave. This makes CCA 10 to generate a sequence of high-voltage sinusoidal pulses, strongly damped and emitted al time intervals TP=TC+TO, amplitude-modulated, which constitute the activation signal the latter by means of its output H and through the high-voltage cable 12 connects to the capacitive application device, same being supported directly on the surface of live system 171 object of treatment which is referred to ground by means of a reference electrode.

Following are the transformation factors defining the functioning of CCA 10:

Ku=voltage amplification factor.

Kf=frequency amplification factor.

Ka=damping factor.

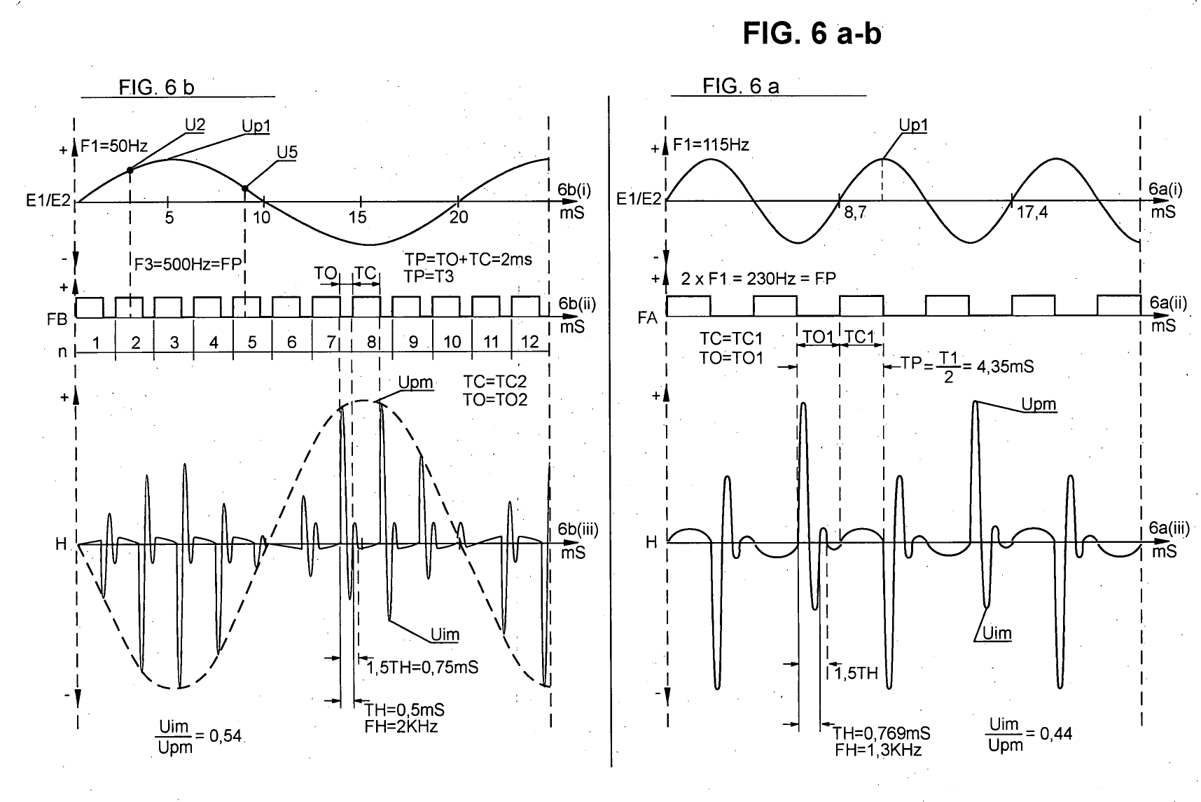

Following are the mathematical equations defining the activation signal for output H of CCA 10 (see FIG. 6b):
w1=2¶F1=sinusoidal wave pulsation of input E.
wh=2¶kfF1=pulsation of output pulse H.
FH=kfF1=frequency of output pulse H.

t=0(zero)---onset of pulse coincident with onset of TP=TO+TC−Ka (FH t)_{—}
Pulse_{—}0≦t≦1,5 TH Uhn=−Ku Un sin(wh t)_{—}
Pause_{—}1,5TH<t<TP=TO+TC Uhn<0,12 Ku Un

Wherein:

Uhn=instantaneous voltage in output H in instant “t”

TH=1/FH=pulsation period at output H.

1,5 TH=time for the first three hemicycles en output H.

n=number of digital pulses counted from the onset of positive hemicycle of the frequency F1 wave.

Un=modulated voltage corresponding to digital pulse number “n”.

Un=Up1 sin [w1 (n TP−TO−_TC)]

Up1=peak voltage for the modulation wave with frequency F1.

The basic operative margins are:

Modulation frequency: F1=5-200 Hz.

Damped sinusoidal pulse frequency: FH=200-3,000 Hz

Damping factor: Ka≧0,9

Digital pulse frequency: Fp=10-1000 Hz

Voltage of output maximum peak: Upm>3.000 Volt

FIG. 6b shows in an exemplary way waveform 6b (i) corresponding to input E of CCA 10 and with frequency F1=50 Hz.

Waveform 6b (ii) corresponding to input FP of CCA and with frequency FP=500 Hz, and at last waveform 6b (iii) corresponding to output H of CCA 10 showing the resulting activation signal with a damped sinusoidal frequency FH=2 Khz, and a damping factor Ka=1,095 which originates a ratio of the second peak and first peak Uim/Upm=0,54.

The neat pulse polarity is defined as the polarity of first peak which in all cases is the one with the highest voltage and as shown in waveform 6b (ii) depends on the polarity of the frequency F1 modulation wave.

The automatic control system of CCA 10 allows to select the functioning values, be it manually or by way of automatic programs for the sequential variation of the peak voltage Upm, frequency FH and polarity of the damped sinusoidal pulses.

CCA 100 Output “Y” forwards to display 16 the functioning values for the parameters adjusted by the manual-automatic control system.

Through its output W timer 15 emits a digital signal which starting from logical state 1 (one) goes to logical state 0 (zero) after time T6 has elapsed. Said signal enters CCA 10 through input W and commands the sequential variation of the parameters adjusted by an automatic programmation.

Output Q of timer 13 starts from logical state 0 (zero) and after a time interval T5 has elapsed reaches logical state 1 (one). This signal enters CCA 10 through input Q and when attaining logical state 1 (one) cancels the emission of the activation signal, thus ending the treatment session.

Timer output Z forwards the elapsed time information to display 16.

Output H of CCA 10 connects through high-voltage cable 12 with metallic plate 13a (the active pole) of application device 13. Metallic plate 13A is covered by plastic housing 13B which rests on live system 171 and maintains a separation distance “d” between the plate and the live system being treated, thus forming a capacitator wherein the dielectric medium is the isolating distance “d” and the plates are on one side plate 13A and on the side the living system 171 grounded to earth by means of an electrode. Therefore upon applying the activation signal for high-voltage pulses there is established a potential or electrical field gradient between conductor plate 13A and the living system being treated, provided with the same waveform as the activation signal and the calculated values of which are:

Ehn=Uhn=Instantaneous value of the electrical field. d

This electrical field established between the active pole and the live system being treated, originates an ionic agitation movement within the live system, by means of the electrical attraction and repulsion forces on the ions in the interior and exterior of the cells; this without inducing any significant heating of the system treated.

The biologically active values of the electrical field are comprised between:

Epm=300-3,000 V/mm wherein:

Epm=Upm/d_=“Peak value” of the electrical field established between the active pole and the surface of the live system,

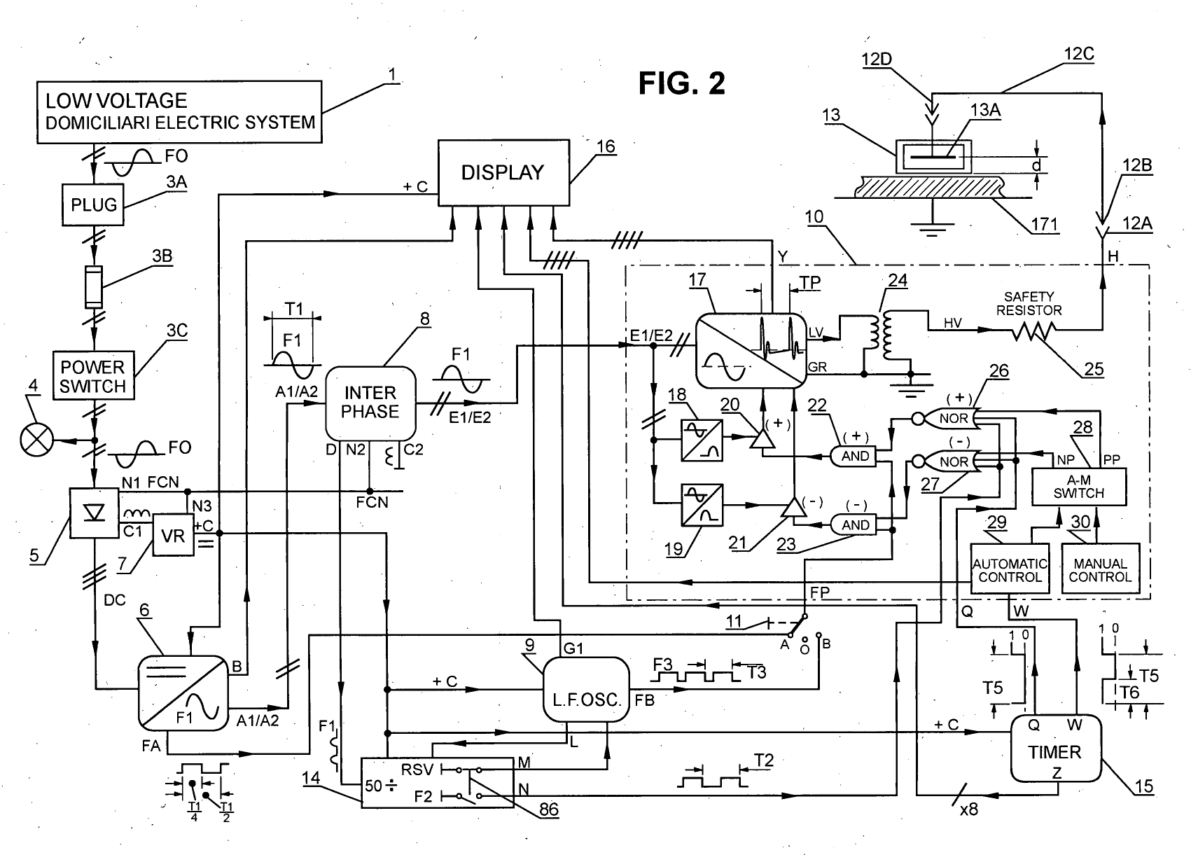

Referring to FIG. 2, which is the preferred embodiment of present invention, same comprises a plug 3A for connecting to mains 1, protection fuse 3B and on/off switch 3C, which feed F0 low voltage low-frequency current to the rectifying circuitry 5; the latter on its turn through its output provides three levels of DC direct voltage to the ondulator circuitry 6 and by means of output C1 provided with full-wave rectified current feeds the voltage regulator source 7, same on its turn and via its output +C provides a stabilized auxiliary control tension for feeding the control circuitry for ondulator 6, oscillator 9, frequency divider 14, timer 15, display 16 and part of the circuitry for controlling the converter amplifier circuit (CCA) 10.

Ondulador circuit 6 converts the direct voltage from the DC connection into an quasi-sinusoidal alternate voltage with a frequency F1 variable from 5 to 200 Hz, and it is this amplitude-modulation wave of high-voltage which connects by means of bipolar output A1/A2 to the interphase circuit 8, the period of this tension wave being T1=1/F1.

Ondulator 6 further provides a digital square wave the period of which is equal to half T1 and which via output FA connects to terminal A of on/off switch 11.

The interphase circuit feeds through its bipolar output E1/E2 an alternate tension of frequency F1, the bipolar input E1/E2 of converter-amplifier 10 thus connecting the circuits of converter 17 and of converters 18 and 19.

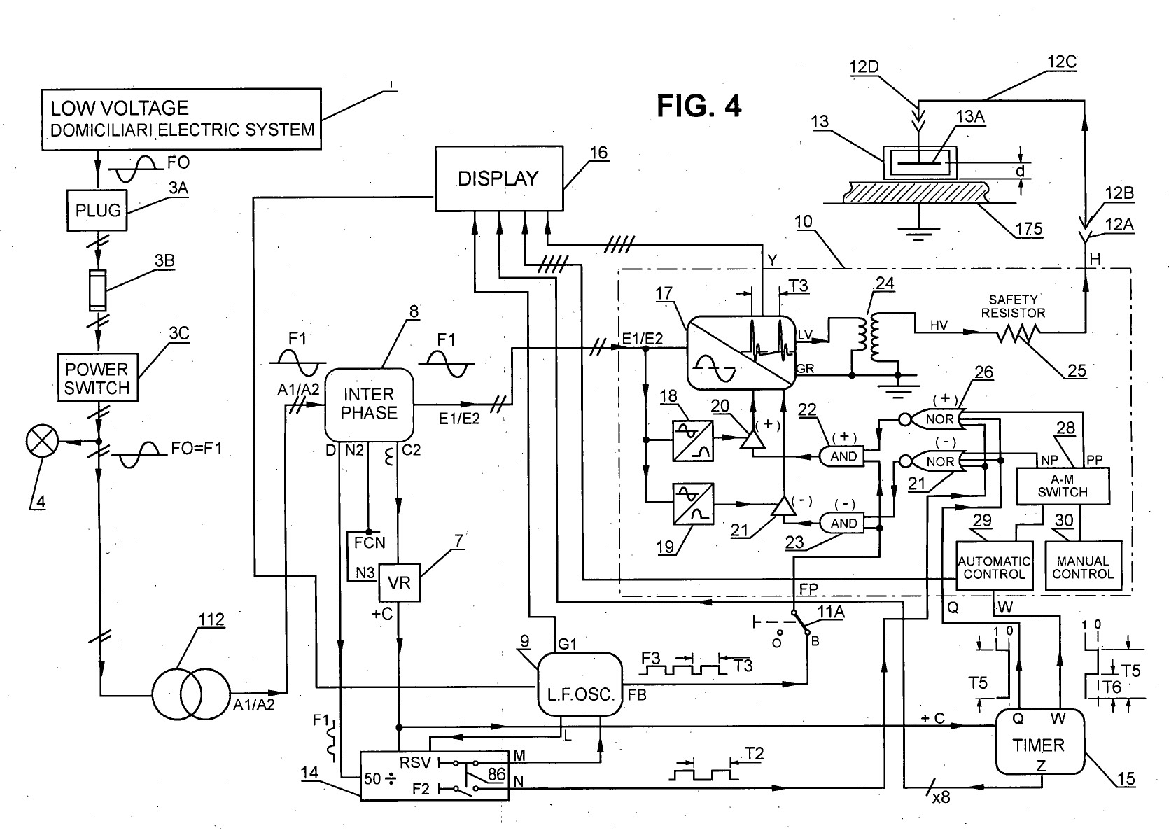

Output C2 of interphase circuit 8 provides a full-wave rectified signal, also of frequency F1, which in this case is not used (it is used in the circuit of FIG. 4).

All the negative power and control poles are connected to a common and floating negative line (not mass-connected) designated FCN. To the latter are connected the negative N1 of rectifier 5, the negative N3 of regulating source 7 and the negatives of the remaining circuits of the apparatus. Connection of line FCN with output N2 of interphase circuit 8 allows to unify the negative terminals of the amplifier circuits 20 and 21 and of gates 22 and 23 of CCA 10 with the remaining circuits.

Interphase circuit 8 provides a rectified half-wave signal which via output D connects to the input of frequency divider circuit 14; said signal has frequency F1. Oscilator circuit 9 is a digital square wave variable frequency F3 generator with 100 pulses per second to 1,000 pulses per second, and the time interval between pulses is T3=1/F3.

Pulse frequency F3 is adjusted to a given value from its own oscillator 9 but further varies from said adjusted point by means of relative sequential variation (RSV) of frequency divider 14 and interconnects to oscillator 9 by means of lines L and M via switch 86.

Frequency F3 pulses of oscillator 9 connect via output FB with terminal B of on/off switch 11.

On/off switch 11 having three positions (A-O-B) connects through line FP one of the inputs of gates AND 22 and 23 of CCA 10; with switch 11 in position “A” enter through line FP the frequency pulses 2 F1 of output FA of ondulator circuit 6; if switch 11 is in position “0”, no pulse sequence enters (circuit is open), and with switch 11 in position “B” frequency pulses F3 from output FB of oscillator circuit enter via line FB.

Frequency divider circuit 14 receives semi-wave signal F1 and on one hand includes the resistive sequence variation system (RCV) and on the other hand divides in 50 the input frequency F1, thus producing a digital square wave with frequency F2=F1/50 wherein the time period between pulses T1=1/F2=50 T1.

Both RSV system and frequency F2 square wave connect before leaving circuit 11 with a bipolar switch 86 such that when output M corresponding to RSV system habilitates output N disables (frequency F2 wave disconnects) and when output N with frequency F2 enables output M disables.

Divider circuit 14 connects via output N with one of the inputs of gates NOR 26 and 27 of CCA 10.

In timer circuit 15 output Q switches from initial logical state zero to logical state 1 (one) once time T5 has elapsed, and connects to a second input of gates NOR 26 and 27 of CCA 10.

Output W transits from initial logical state 1 (one) to logical state 0 (zero) once time T6 has elapsed the latter being approximately equal to half time T3. Said output W connects with automatic control circuit 29 of CCA 10.

Output Z of 8 bit timer circuit 15 provides the signal indicating the time elapsed to display 16. Ondulator 6 via its output B and oscillator 9 via its output G1 forward the information on their respective working frequencies to display 16.

Display 16 is mounted in the frontal part of the equipment and houses the commands for all the switches and push-buttons of the apparatus, as well the mechanical, luminous and digital signalizations of the various circuits of the apparatus.

Converter-amplifier circuit (CCA) 10 comprises a alternate current converter 17 producing low-tension voltage sinusoidal pulses, strongly damped and amplitude-modulated by the same frequency F1 input sinusoidal wave; a semi-wave counter-phase converter 18 providing feeding voltage to a current amplifier 20 and to AND gate 22; a half-wave in-phase converter 19 which provides feeding tension to current amplifier 21 and to gate AND 23. In such a way when alternate tension wave (which may be sinusoidal or quasi-sinusoidal) of frequency F1 from output E1/E2 of interphase circuit 8 is crossing its positive hemicycle, it is in condition only to operate amplifier 21 and gate 23, whereas when the alternate tension crosses its negative hemicycle it can operate only amplifier 20 and gate 22.

The output of gate 22 connects to the input of amplifier 20 and the output of the latter connects to converter 17 in such a way that for every pulse emitted through gate 22 converter 17 emits a damped sinusoidal pulse with a positive initial peak (under such conditions the sinusoidal wave will go through its negative cycle). The whole of amplifier 21 and gate 23 works in a similar manner except that the initial peak of damped sinusoidal pulse will have a negative polarity (under such conditions the sinusoidal wave will cross its positive hemicycle). The pulse tension peak value will depend from the duration time of the output pulse from gates AND 22 and 23 and from the mean value resulting from the instantaneous values of frequency F1 sinusoidal wave tension which were encompassed during said pulse.

The damped sinusoidal pulses connect via output LV of converter 17 with one of the terminals of the primary of booster transformer 34. Output GR of converter 17 and the other terminal of the primary and its homologue secondary of transformer 24 are mass-connected, thus from output terminal of HV secondary HV of transformer 24 are obtained the high-voltage sinusoidal damped pulses. This HV output connects to a safety resistor 25 producing a very low voltage drop due to the passage of capacitive microcurrents from capacitive application device, 13, but which when faced with a non intentional direct contact limits the output current values to levels harmless to humans and superior animals.

Resistor connects through line H of CCA with high-tension socket 12A mounted in the equipment housing. High tension plug 12B, high-tension cable 12C and female connector 12D connect the capacitive application device 13. Distance d is the separation between the active metallic electrode 13 A and live system 171 which is grounded; in such a way the established potential gradient is E=U/d wherein U is the applied tension; in this case the tension applied is the activation signal constituted by the high-voltage amplitude-modulated pulses, the time period Tp between one pulse and the other being given by the time period between the pulses entering gates AND 22 and 23 via line FP and are enabled by gates NOR 26 and 27 when the digital outlets of same are in logical state 1 (one) and disabled when in logical state 0 (zero). The output of gate NOR 26 connects to the input of gate AND 22 and output NOR 27 connects with input of gate AND 23.

In such a way the outputs of gates NOR 26 and 27 will depend from the state of their inputs; if their three inputs are in logical state 0 (zero) their output will be 1 (one) and therefore be enabling; if any input is in logical state 1 (one) its output will be in state 0 (zero) and therefore disabling.

One of inputs of gate NOR 26 and 27 is the connection with output N of divider circuitry 24 which leads the frequency F2 pulse sequence via the closing of switch 86; in this case the emission of the high-voltage pulse activation will be intermittent and therefore this intermittence will have frequency F2.

The second input of gates AND 26 and 27 is output “Q” of timer circuit 15 which after a time T5 has elapsed transits to logical state 1 (one) disabling indefinitely the emission of the activation signal.

The third output from gate NOR 26 connects to line PP of manual/automatic switch 28 and the third input of gate NOR 27 connects to line NP of switch 28. When line PP is in logical state 1 (one) it disables the high-voltage pulses having an initial positive peak, the only pulses now being emitted are those with an initial negative pulse. When line NP is in its logical state 1 (one) the opposite happens to previously described.

Lines PP and NP may both be in logical state 0 (zero) (enabling) but never both in logical state 1 (one) simultaneously.

In its “manual” position manual-automatic switch 28 allows the passage to lines PP and NP of orders proceeding from the manual control circuit 30 only. When switch 28 is in its “automatic” position, it only allows the passage of orders proceeding from automatic control circuit 29.

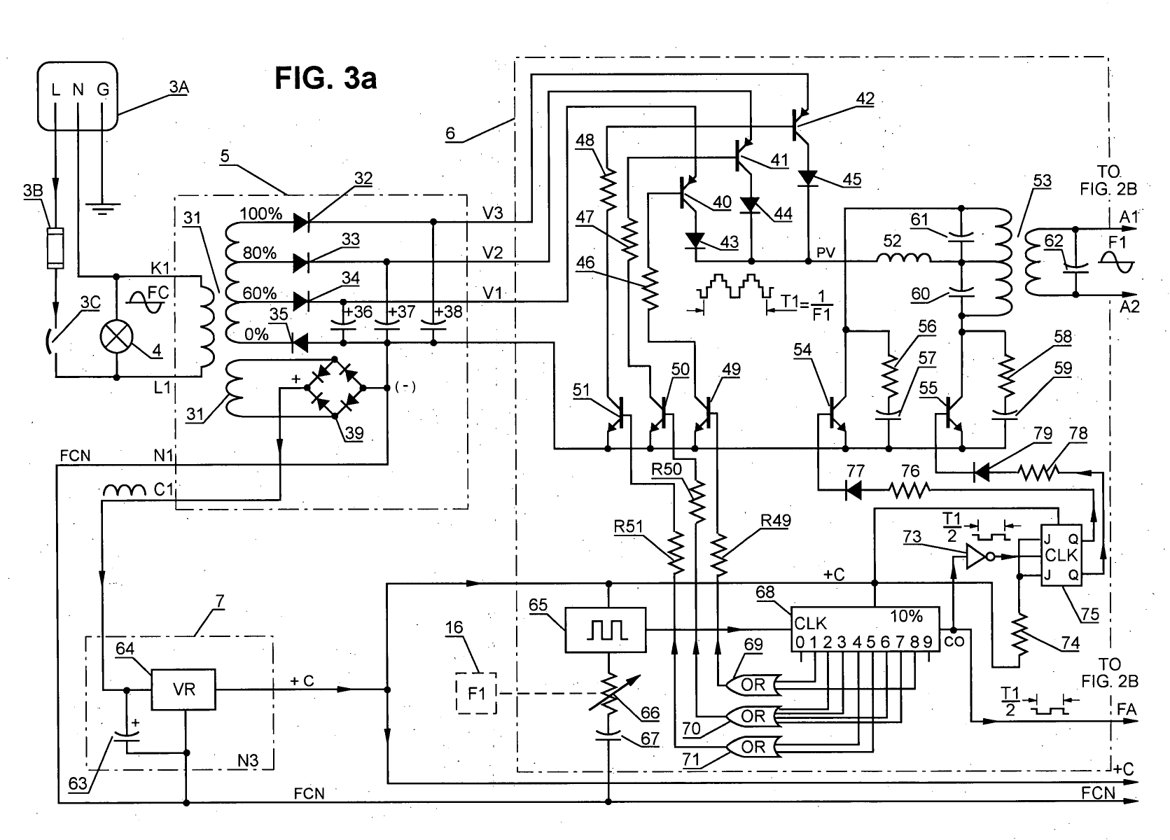

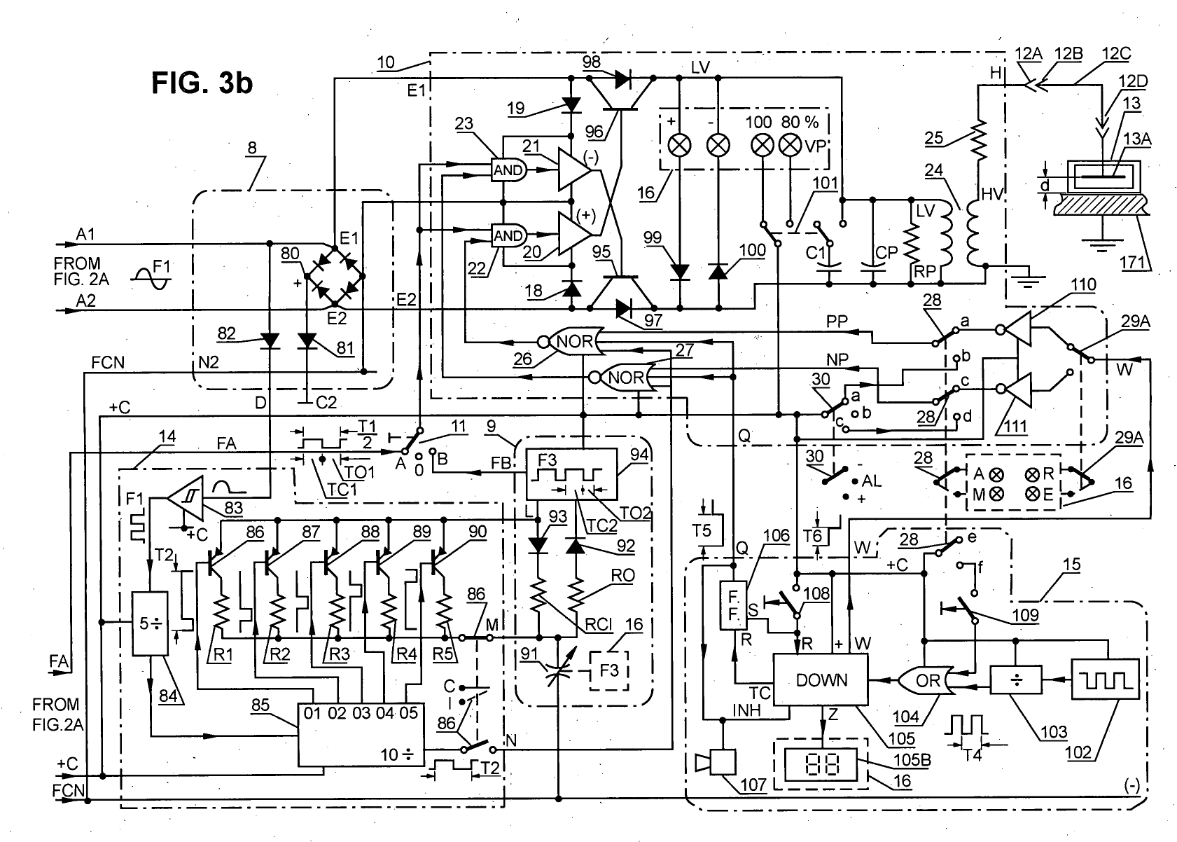

FIGS. 3a and 3b illustrate more clearly and precisely the composition and functioning of the blocks shown at FIG. 2.

In FIG. 3a plug 3A for connecting to the mains 1 the terminal L corresponding to active pole (live pole) of the main connects to protection fuse 3B, terminal B corresponding to neutral pole connects to neon lamp 4 and to terminal K1 of the primary winding of transformer 31 corresponding to rectifier circuit 5, and terminal G corresponding to ground connects to the mass of the apparatus.

Protection fuse 3B connects to switch 3C which commands the global powering of the equipment, connecting or disconnecting the electrical power from the active pole of the mains.

Said switch connects to the other terminals of lamp 4 and with terminal 11 of primary winding of transformer 31. Lamp 4 shows apparatus is powered.

Rectifier circuit 5 includes transformer 31 which is a monophasic transformer with three windings: a primary input and two secondary ones, the power secondary having three tension levels (60%, 80%, 100%) and the other secondary is the one feeding the auxiliary consumptions.

At the power secondary the 100% output connects to the anode of rectifier diode 32 and the cathode to the positive terminal of filtration capacitator 38, thus attaining a continuous tension 100% of the nominal (rated) voltage in line V3. The 80% output connects to the anode of diode 33 and the cathode to the positive terminal of capacitator 37 and likewise the 60% output connects to diode 34 and to capacitator 36, thus conferring lines V1 and V2 80% and 60% respectively of the nominal tension; the negative pole of said continuous tensions is in common and is attained at anode of diode 35 which permits to connect its cathode with terminal 0% return of the currents circulating through lines V1, V2 and V3. Said negative power terminal joins the negative terminal of rectifier bridge 39 thus originating line N1 which connects to the line of FCN (floating-common-negative) the latter being the general negative of the apparatus.

Diode bridge 39 connects to the auxiliary secondary winding, transformer 31 and originates full wave rectification line C1 which connects to filtering capacitator 63 and to voltage regulator of the regulated source circuit 7, the negative terminal N3 of said bridge connects to the floating general negative (FCN), line +C constitutes the line feeding a continuous stabilized positive polarity control tension to the digital logic of the apparatus' circuitry. Ondulator circuit 6 includes a power circuit and a logical digital auxiliary circuit. The ondulator circuit receives power from lines V1, V2 and V3 of rectifier 5 and connect to the emitters of PNP power transistors 40, 41 and 42, respectively. The bases of said transistors connect through resistances 46, 47 and 48 with the collectors of auxiliary NPN transistors 49, 50 and 51 respectively and collectors of transistors PNP connect to the anode of rectifying diodes 43, 44 and 45 respectively: the cathode of said three diodes join into a common point PV which will have the electrical tension of transistors PNP 40, 41 or 42 which are enabled for conducing and may adopt values of 0%, 60%, 80% or 100%. This common point PV connects to a terminal of smoothening inductance 52 and its other terminal connects to the common point joining the filtration capacitors 60, 61 and the middle point of transformer's 53 primary winding.

The ends of said winding connect on one side with capacitator 61, resistor 56 and collector of power NPN transistor 54; the other end of transformer 53 primary winding connect to capacitator 60, resistor 5 and the collector of power NPN transistor 55. Resistor 56 serially connected with capacitator 57 and resistor 58 serially connected with capacitator 59 protect transmitters 54 and 55 respectively when transiting from a conductive state into a powerless state.

The emitters of transistors 54 and 55 and of auxiliary transistors 49, 50 and 51 connect to the power negative line.

Line +C of regulating source 7 provides auxiliary feeding to oscillator 65, to divider counter 68, flip-flop JK 75 to gates OR 69, 70 and 71 and inverter 73, which constitute the auxiliary circuit of ondulator 6 digital logic.

Oscilator 63 is a square-wave variable frequency generator with a time base (resistance-capacity) RC given by variable resistor 66 and capacitor 67; the square-wave frequency varying by resistor 66. Oscilator 65 output connects to CLK of divider counter 68 which is a decade counter of the JONSON type with 5 (five) stages wherein outputs 0 and 9 transit sequentially from logical state one for every pulse of oscillator 65, and via output CO a square wave is emitted for every 10 pulses from oscillator 65, i.e., the input frequency is divided by 10.

Outputs 0 and 9 of counter 68 stay free, outputs 1 and 8 connect to inputs of gate OR 69, outputs 2, 3, 6 and 7 connect to the inputs of gate OR 70 and outputs 4 and 5 connect to the inputs of gate OR 71. The outputs of gates OR 69, 70, 71 connect with bases of transistors NPN 49, 50, 51 respectively via resistors R 49, R 50 and R 51 respectively. This circuitry causes that when output 0 crosses through logical state 1 transistors PNP 40, 41 and 42 are shutoff and therefore tension on point PV, will be zero.

When the output 1 transits logical state one, transistor PNP 40 will become enabled and the tension on point PV, will be 60% of the rated (nominal) one, when outputs 2 and thereafter 3 cross logical state one transistor PNP 42 enables and the tension of point PV will become 80% of the nominal tension, thereafter they will transit to logical state one the outputs 4 and 5 thus enabling transistor PNP 42 and the tension of point PV will become 100% of its rated value; thereafter outputs 6 and 7 come up and the tension in PV will become 80% of its rated value, thereafter the output 8 and the tension in PV will become 60% of its rated value, thereafter follows output 9 which because of not being connected will lead point PV to zero. This cycle repeats indefinitely thus causing in point PV an unidirectional tension stepped variation.

On the other hand output CO connects via inverter 73 to CLK of descending counting FLIP-FLO JK 75 and the terminals J and K connect via resistor 74 to line +C. Thus every 10 pulses of oscillator 65 a stepped tension wave is created at point PV ranging 0 to 100% and returns to tension 0 and simultaneously, at every onset of this wave, outputs Q and complementary outputs Q, invert their logical states causing transistors NPN 54 and 55 to alternate periodically. The base of transistor 54 connects via diode 77 and resistor 76 with terminal Q of FLIP-FLOP 7 and the base of transistor 55 connects via diode 79 and resistor 78 with complementary terminal Q of FLIP-FLOP 75, this ensuring that when a transistor is conductive the other one stays shutoff.

When transistor 54 is conductive, the stepped tension connects into a branch of the primary winding of transformer 53 and when transistor 55 is conductive, the stepped tension connects into the other branch of primary winding thus originating in the secondary winding of transformer 53, an alternate tension which due to the effect of inductance 52 and capacitors 60, 61 and 62, is virtually sinusoidal. In such a way a full cycle of quasi-sinusoidal frequency F1 is obtained for every 20 pulses of oscillator 65.

This quasi-sinusoidal tension connects via lines A1 and A2 with interphase circuit 8 of FIG. 3b, line A1 connects to the cathode of diode 82 and to terminal E1 of diode bridge 80; line A2 connects to terminal E2 of diode bridge 80. The negative terminal (−) of bridge 80 connects via line N2 with the negative connection of FCN (floating common negative) from FIG. 3a thus unifying the negative pole of the auxiliary circuit for digital control logic pertaining to converter-amplifier circuit (CCA) 10.

The positive terminal (+) of diode bridge 80 connects to the cathode of diode 81 and its anode connects to line C2 thus creating in said line a full ware rectified tension which in this case lacks use.

The anode of diode 82 connects via line D a rectified semiwave tension signal with frequency F1 to the input of converter buffer Schmit circuit 83 of frequency divider circuit 14. The digital logic of divider circuit 14 is fed via the positive polarity tension line +C.

The converter buffer Schmit circuit transforms the input semiwave signal into a digital frequency F1 signal and connects it to the input of a frequency divider 84 which divides frequency F1 by 5. The output of divider 84 connects to the entry of a counter-divider 85 the outputs 01 to 05 of which connect to the bases of transistors PNP 86 to 90 enabling them sequentially every time the corresponding output transits to logical value zero. Said sequence is shown in diagram form 7, where waveform 7 (i) represents the frequency F1 alternate tension signal arriving at terminals E1 and E2 of (CCA) 10. Waveforms 7(ii) to 7 (Vi) corresponds to outputs 01 to 05 of counter-divider 85 where it can be observed that the outputs cross sequentially through zero every 10 time periods of frequency F1 wave and this repeats cyclically till the end of timing when output Q of timer circuit 15 transits to logical state 1 (one), once time T5 has elapsed as shown in waveform 7 (Viii). The full sequence lasts a time T2 which will corresponds to 50 periods of original wave F1.

Waveform 7(Vii) corresponds to the output CO of counter-divider 85, same being a square digital symmetric wave of period T2, i.e. the frequency of this wave will be: F2=F1/50.

As mentioned above, every output 01 to 05 acquires logical state 0 (zero) and enables the corresponding transistor, e.g. when output 01 transits to 0 (zero) transistor 86 is conductive and connects resistance R1 via contact M of switch 86 in parallel with the branch of diode 93 and resistance RC1 of oscillator circuit 9. The addition of said resistance modifies the oscillator time base and therefore alters frequency F3 value. According to the values given to resistances R1 to R5 it is possible to attain sequential variations of the adjusted base frequency base which cover from no variation at all in the case that all the resistances R1 a R5 are equal, till 5 possible variations in case all resistances have different values. When switching switch 86 from position C (continuous emission) to position 1 (INTERMITTENT EMISSION) output CO of divider-counter 85 is connected with terminal N of switch 85 and feeds with square frequency F2 wave signal one of the inputs of gates NOR 26 and 27 of CCA-10 thus causing the intermittent functioning as previously explained. Therefore the apparatus generates high-voltage pulses for half time T2 wherein the wave crosses logical state 0 (zero) and will not emit high-voltage pulses for half time T2 wherein the wave crosses logical state 1 (one).

During the intermittent functioning there is no sequential variation of pulse frequency F3 of oscillator circuit 9 as terminal M of switch 86 disconnects resistances R1 to R5 and, therefore oscillator 9 works with the adjusted frequency value only by means of regulable capacitor 91.

Oscillator circuit 9 includes a digital square wave variable frequency generator with time base RC (RESISTANCE-CAPACITY) wherein the time during which the wave crosses logical state 1 (one) TC2—also called mark time—depends from the value of resistance TC1 and the adjusted value in regulable capacitator 91 while the time during which the wave crosses logical state 0 (zero)—also called space time—depends from the resistance (RO) value and from the capacitor 91 value. Diodes 93 and 92 determine one and another case by their sense. The charging time of capacitator 91 determines the marking time and the discharge time of said capacitator determines the space time. The sequential resistance variation caused by connection of terminal M of switch 86 originates a sequential variation in the charging time of capacitator 91 and therefore varies marking time TC2 only and space time TC2 remains unchanged.

Base frequency F3 is fixed with capacitator 91 and indicator display 16 shows the adjusted value.

Output line FB connects frequency F3 digital square wave of pulse generator 94 to terminal b of on/off switch 11. Line FA connects frequency 2×F1 digital square wave of output CO of counter-divider 68 of FIG. 3a with terminal A of on/off switch 11.

As previously explained the position of this on/off switch 11 gives three (3) possible alternatives. In position “0” converter-amplifier 10 does not emit high-voltage pulses. In position “A” via line Fp connect the inputs of gates AND 22 and 23 the frequency 2×F1 square wave the time period of same between two successive pulses is T1/2. As shown in waveform diagram in FIG. 6a, wherein waveform (a) represents frequency F1 alternate tension arriving at terminals E1 and E2 of (CCA) 10; in an exemplary way F1=115 Hz. Terminal E1 connects to anodes 19 and 18 and to emitter of transistor 96, whereas terminal E2 connects to the anodes of diodes 18 and 97 and to emitter of transistor 95. Waveform 6 a (ii) represents the square wave which being transmitted from line FA enters via switch 11 in position A and line Fp into gates AND 22 and 23 of CCA 10.

For every hemicycle of waveform 6a (i) there is a mark and a symmetrical space of waveform 6a (ii). Under said conditions when gate outputs NOR 26 and 27 are in logical state 1 gates AND 22 and 23 will be enabled; therefore into current amplifiers 20 and 21 will enter simultaneously equivalent pulses of waveform 6a(i).

When hemicycles of waveform 6a (i) are positive terminal E1 will be positive with respect to E2, and therefore diode 19 will function as an in-phase halfwave converter as already explained for FIG. 2 and therefore gate AND and amplifier 21 will receive auxiliary control feeding. Waveform pulses 6a(ii) are amplified and converted by amplifier 21 into current pulses with an amplitude such that when being applied to the base of potency transistor NON 95 the possibility that during mark time TC1 transistor 95 transits to a conductive state and during space time TO1 same will stay shutoff. At the same time power transistor NPN 96 will stay shutoff as diode 18 functioning as an out-of-phase-halfwave disables gate AND 22 and amplifier 20, in this hemicycle transistor 96 has its emitter more positive than its base.

During the transistor 95 conductive state (time TC1) electrical current will flow from transformer 53 via line A1, terminal E1, through diode 98 which will stay directly polarized, and this current will charge parallel circuit (RLC) comprised of capacitator CP, resistance RP and inductance of primary winding of booster transformer 24, for which the capacitive charge of applicator device 13 is very low, and therefore said inductance corresponds practically to the one of primary winding of transformer 24 with its secondary winding open. Electrical current will return to transformer 53 via transistor 97, terminal E2 and line A2 thus closing the potency electrical circuit.

Upon charging, the tension of circuit RLC will be the same as of primary winding and will reflect amplified in terminal HV of secondary winding of transformer 24. The electrical tension values of terminal HV are slightly above those of terminal H—as during normal operation the capacitive charge of application device 13 is low and the tension drop in resistor 25 is very low. The electric tension values grounded of terminal H are represented in waveform 6a(iii) wherein it can be appreciated that during time interval TC1 the positive land represents the tension variation during the charging of circuit TLC seen from the high-voltage side. Thereafter, once time TC1 has elapsed and crosses abruptly transistor 95 to the shutoff stage, the current which was flowing through primary winding of transformer 24 due to the inductive effect with secondary open causes an abrupt peak with negative polarity (opposite to the polarity of charging land) and, this being a RLC circuit, the impulse adopts a strongly damped sinusoidal oscillation waveform.

The subsequent oscillations will be cancelled with the onset of following mark time which will be coincident with the onset of the negative hemicycle of waveform 6a(i) (terminal E2 is more positive than terminal E1) and therefore gate AND 2 will function and amplifier 20 this will leading transistor 96 to its conductive state and therefore now electrical current will flow from transformer 53 via line A2 terminal E2 through diode 97, charging parallel circuit RLC in inverted form with respect to prior case and the electrical current will return via transistor 96, terminal E1 and line A1 to transformer 53, and electrical circuit closes during negative hemicycle of frequency F1 alternate tension. As can be appreciated in waveform 6a(iii) the lands and peaks during the negative hemicycle are equal but with signs opposed to those of the positive hemicycle. The grounding of the windings of transformer 24, resistance RP, capacitors CP and C1, anode of diode 100, cathodes of diodes 97 and 99 and transistor collector 97 by means of terminal G of plug 1 when connecting to ground line, set forth the ground reference for output pulses H.

Waveform 6a(iii) represents the high-voltage pulse activation signal for the operative condition of on/off switch 11 in position “A” wherein there is only one damped sinusoidal pulse for every hemicycle of waveform 6a(i).

Waveform 6a(i) is the low-frequency modulation quasi-sinusoidal wave generated by ondulator 6, and waveform 6a(ii) represent the low voltage high-frequency pulses.

Polarity of damped pulse is defined as the polarity of first peak which is the greatest of all.

By definition, the peak maximum tension equals Upm and the maximum inverted peak tension equals Uim.

Pulsation period is defined as TH and pulsation period is defined as FH=1/TH.

Damping relation is defined as Uim/Upm<1.

For the example in Figure following ensues:

| | |
|---|---|
| F1 = 115 Hz. | T1 = 8.7 m sec. |

TC1 = TO1 = T½ = 4.35 m sec.
TH = 0.769 m sec.
FH = 1.3 Khz
Uim/Upm = 0.44

With on/off switch 11 being positioned in “B” the pulses from output FB of pulse generator 94 will enter CCA 109 via line FP; the waveforms ensuing from this alternative are shown at FIG. 6b.

Waveform 6b(i) is the quasi-sinusoidal alternate modulation wave arriving at terminals E1 and E2 of (CCA) 10 as in prior case, but in this example F1=50 Hz is adopted.

Waveforms 6b (i) are the high-voltage frequency F3 pulses of output FB of digital pulse generator 94 for which in this case frequency F3=500 Hz is adopted for which the periodic interval between pulses is T3=2 ms=TC2+TO2 wherein TC2=mark time and TO1=space time; the functioning of this alternative is equal to prior case of FIG. 6a the only difference being that for every hemicycle of waveform 6b(i) there is more than one waveform pulse 6b(ii), in this example there are 5 (five) pulses. Thus high-voltage activation signal in terminal H corresponding to waveform 6b(iii) will have more than one damped sinusoidal pulse per hemicycle, which in this case will be 5 (five) cycles. It can be observed that during mark time TC2 a charge land of parallel circuit RLC and during space time TO at onset of damped oscillation, wherein the high-voltage pulses are amplitude-modulated by waveform 6b(i). This modulation is due to the fact that the value of current that will circulate through the primary winding of transformer 24 will depend from the mean value of the tension applied on parallel circuit RLC during mark time TC2, and this mean value will be different for the various mark times of a given hemicycle.

Waveform 6b(ii) represents the high-voltage pulse activation signal amplitude-modulated by low-frequency quasi-sinusoidal wave of waveform 6b(i) for the operative condition of switch 11 in position “B”.

For the example shown in FIG. 6b:

| F1 = 50 Hz | T1 = 20 msec |
|---|---|
| F3 = 500 Hz | T3 = 2 msec |
| TC2 + TO2 = T3 = 2 msec | |
| TH = 0.5 msec | |
| FH = 2 Khz | |
| Uim/Upm = 0.54 | |

TP being the periodic interval between the damped sinusoidal pulses. Following are the margins of the apparatus operative values:

Alternative a (Switch 11 in position “A”):

| | |
|---|---|
| Modulation Frequency | F1 = 20-200 Hz |
| High-voltage Frequency pulses | Fp = 40-400 pulses/sec. |
| Damped frequency Pulsation | FH = 200-2000 Hz |
| Damped pulsation Period | TH = 5-0.5 msec |
| Damping Ratio | Uim/Upm = 0.2-0.5 |
| Maximum Peak Voltage | Upm > 3000 Volt. |
| Potential Gradient or biologically | E = 300-3000 Volt/mm |
| Active Electrical Field | |

Alternative b (switch 11 in position “B”):

| | |
|---|---|
| Modulation Frequency | F1 = 5-50 Hz |
| High-voltage frequency pulses | Fp = F3 = 100-1000 pulses/sec. |
| Damped pulsation frequency | FH = 1500-3000 Hz |
| Damped pulsation Period | TH = 5.55-3.70 msec. |
| Damping Ratio | Uim/Upm = 0.666-0.333 msec |
| Maximum Peak Voltage | Upm > 3000 Volt. |
| Potential Gradient or biologically | E = 300-3000 Volt/mm |
| Active Electrical Field | |

In both alternatives the RMS value for the electrical current capacitively induced has a power level which under no circumstances causes any significant heating of the system under treatment.

Capacitive application device 13 is placed on the system to be treated, 171, be it cell cultures, bacteria cultures, fungi cultures or for its biomedical use on patient's injured part. The system to be treated is grounded by means of an electrode and thus the potential gradient or electrical field established between the active electrode of applicator 14A and the surface being treated will be: Em=Upm/d wherein:

Em=Potential gradient or maximum electrical field established

Upm=maximum peak voltage applied

d=distance between active electrode and surface to be treated

Neon lamps (+) and (−) in display 6 will light only for peak values of low-tension side activation signal, and therefore lamp (+), serially connected with diode 99, upon lighting will reveal the presence of positive pulses in the activation signal, whereas lamp (−), serially connected with diode 100, upon lighting will reveal the presence of negative pulses.

Switch 101 is a bipolar (2) switch with two positions. In the position corresponding to Capacitator C1, the lamp being disconnected, 100% of display 16 receives through switch 100 feeding from line +C, this indicating that the equipment will emit 100% of the maximum possible peak value. Capacitor C1 being connected via switch 101 in parallel with CP, RP and the primary of transformer 24, the equipment will emit 80% of the maximum tension possible. Under such conditions it will receive power from line +C; lamp showing 80% means that the equipment will emit 80% of the nominal (rated) peak tension,

The manual control of polarity is done using on/off switch 30 having one pole and 3 (three) positions:

(−) emission of pulses with initial negative peak

AL alternate emission, positive and negative pulses

(+) emission of pulses with initial positive peak

Selection of polarity by manual or automatic control is done by means of on/off switch 28 provided with 4 (four) poles 2 (two) positions.

A—Automatic control of polarity

B—Manual control of polarity

The manual position of switch 28 is shown on display 16 by means of visual signal M. If switch 30 is in position (−) its connects via terminal “a” of switch 30 and terminal “b” of switch 28 line +C with line PP carrying positive control tension to the third input of gate NOR 26 y therefore establishing a digital 0 (zero) to the output of latter and disabling gate AND 22, this canceling the pulse generation which entered amplifier 20 and therefore canceling the emission of high-voltage pulses provided with an initial positive peak, that is to say there will be emitted only those pulses with an initial negative (−) value, Switch 30 being in position 30 in position (+) connects line +C through terminal C of switch 30 and terminal “d” of switch 28 with line NP carrying positive tension control to input of gate NOR 27, thus disabling gate AND 23 y thus canceling high-voltage pulses of an initial positive voltage; if switch is in position “AL” line C will no connect line +C to any of both (2) gates NOR 26 and 27 and therefore they will be an emission of negative and positive pulses, the alternate activation signal being as shown in waveform 6a(iii) or 6b(iii).

Gates NOR 26 or 27 and inverters 110 and 111 receive feeding control tensions via line +C, the latter also feeding the electronic logic of timer circuit 15.

Timer circuit 15 includes an oscillator 102 which is a stable digital square frequency generator the output of which connects to one of the inputs of gate OR 104 via the frequency divider 103 which delivers a digital square wave with periodical intervals between pulses T4=1 minute, i.e. it generates one pulse per minute. The output of gate OR 104 connects with a descending programmable counter 105 which upon equipment being powered and receiving auxiliary control tension through line +C a number of 20 minutes is preselected and defined in counter 105, said number being shown via 8 bits line Z on digital reading device 105 B of display 16. For each impulse from OR gate counter 105 continues counting descendently a time T5 of 20 minutes; reader 105B finishes indicating number “00”. Output TC of counter 105 connects to reset entry R of a set—reset flip-flop 106. Output signal Q from flip-flop 106 which originally was in logical state 0 (zero) transits now to logical state 1 (one) thus controlling on one hand gates NOR 26 and 27 and disabling the frequency input via line FP towards amplifiers 20 and 21 and therefore canceling the generation of the activation signal of high-frequency pulses (end of emission). On the other hand output Q of flip-flop 106 connects to a buzzer 107 which emits an acoustic signal and to inhibition control INH of counter 105 this totally inhibiting the activation and counting actions.

Upon being depressed digital pulse button 108 applies a positive tension pulse of line +C to entry set S of flip-flop 106 and to reset control R of counter 105, in such a way that the prefixed number of 20 minutes can be newly defined in counter 105 crossing output Q of lip-flop 106 to logical state 0 (cero), this again enabling the emission of the activation signal and canceling the acoustical signal from buzzer 107 and reinitiating a new 20 minutes timing cycle.

When switch 28 is in its manual position digital pulse button 109 allow to input positive tension pulses from line +C via line “f” of switch 28 to the input of gate OR 104 and thus to select a timing time less than 20 minutes. This operation is disabled when switch 28 is its automatic position. In said latter condition inverter 110 connects via terminal “a” of switch 28 with line PP, and inverter 111 connects via terminal C of switch 28 with line NP, this enabling the automatic control of the activation signal polarity. Switch 28 A is a 2 (two) pole 2 (two) positions on/off switch.

R=tissular regeneration

E=contractures and edemas

One pole connects the visual indication R and E on indicator board 16 and the other pole preselects the inputs of inverters 110 or 111 through connecting them with output W of counter 105. At the start of timing said output W is in local state 1 (one) and after a time T6 T5 has elapsed transits to state 0 (cero) as shown in waveform diagram 7 (1X) when switch 29A is in position R during the first timings stage inverter 111 receives a digital 1 (one) from output W and the output of inverter 110 is in logical state 0 (zero) and therefore the activation signal for the high-voltage pulses comprises alternate positive and negative pulses. After a time T6 and having changed its state, output W of counter 105 line PP transits to logical state 1 (one) disabling gate NOR 26 and canceling the positive pulses of the activation signal till the end of timing when time T5 has elapsed; the effect of output Q of flip-flop 106 will also cancel the negative pulses of the activation signal, thus ending the application.

Switch 29 A being in position E during the first stage, the activation signal will be constitute alternate positive and negative pulses. Once time T6 has elapsed the negative pulses will cancel and the activation signal will comprise positive pulses only till the end of timing.

The positive pulse of activation signal is defined a that damped sinusoidal pulsation having an initial positive peak and the negative pulse of which is that damped sinusoidal pulsation having an initial negative peak as shown at waveform 6a(iii) and 6b(iii). The positive pulses of the activation signal will come up when the modulation wave represented in waveform diagram 6a(i) and 6b(i) crosses its negative hemicycle and the negative pulses of the activation signal come up in the positive hemicycles of the modulation wave.

Referring now to FIG. 4, the difference with diagram of FIG. 2 is that the feeding from mains 1 via plug 3A, fuse 3B and activation switch 3C arrives directly to the primary winding of a transformer 112 which receives low-voltage low-frequency from mains F0. In this case frequency F1 of the modulation wave will be directly F0, and therefore F1=F10=mains frequency, and the apparatus will operate with a modulation frequency only.

Output of secondary winding of reducing transformer 12 connects—likewise as in diagram of FIG. 2—via line A1 and A2 with interphase circuit 8. The regulated tension source 7 is fed via line C2 of interphase circuit 9. The remainder of the circuits are exactly the same as described in FIG. 2 and their similar ones en FIGS. 3a and 3b, with the exception of on/off switch 11a which in this case has two (2) positions, 0=without emissions and B=emission of pulses from oscillator 9 via connection of line FB with line FP by means of terminal B of switch 11.

For this second embodiment of the apparatus according to FIG. 4 the alternative exists of FIG. 6b with the 50 Hz modulation in waveform 6b(i), low-voltage waveform sequence 6b(ii), and the activation signal of low-voltage waveform 8b(iii).

When equipment is connected to F0=60 Hz mains frequency modulation F1 will become 60 Hz too.

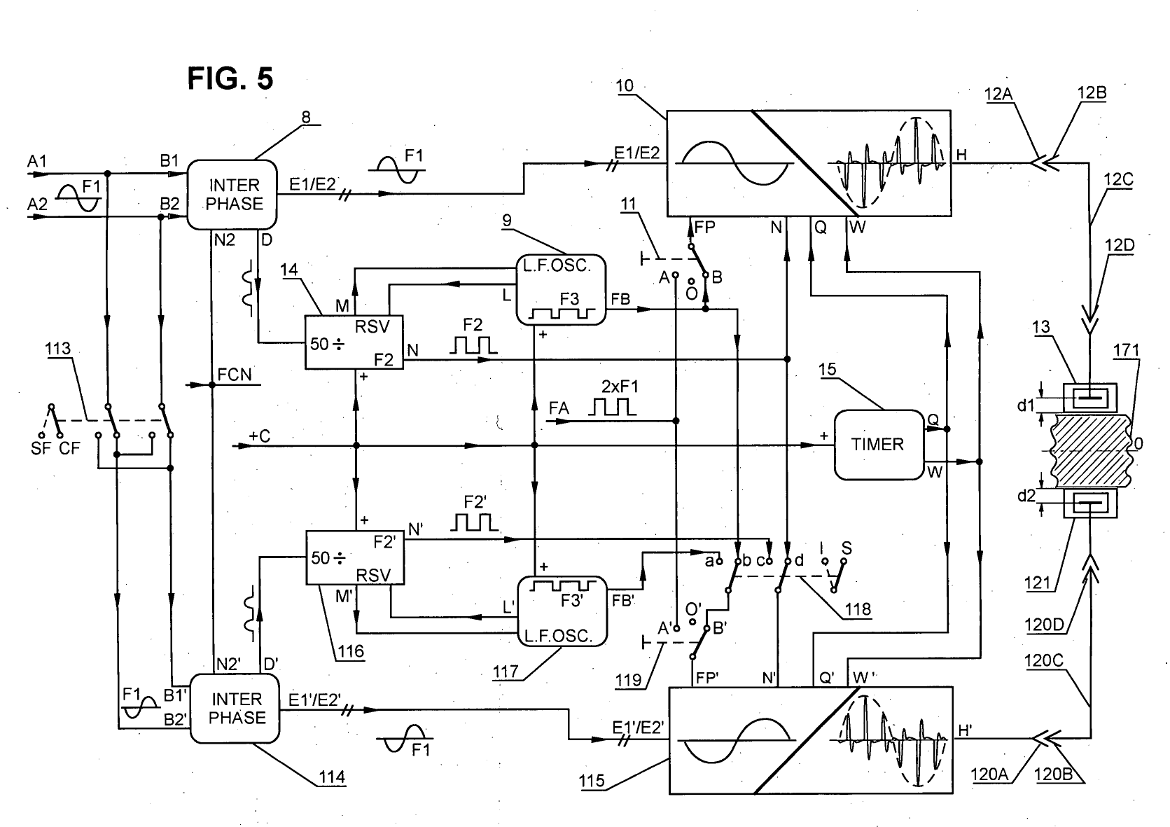

En FIG. 5 lines Aq1, A2, FCN, +C and FA are equal and are obtained in the same way and with the same components as the ones shown at FIG. 2, FIG. 3a and FIG. 3b. In this diagram there are two (2) high-voltage pulse generators.

The first generator includes an interphase circuit 8, an oscillator circuit 9, a converter-amplifier circuit 10, an on/off switch 11, a frequency divider circuit 14, a high-voltage socket 12 A, a high-voltage connection device comprising a plug 12. B, a cable 12C, a connector 12D and a capacitive application device 13. Their composition and functioning are the same as the ones shown in FIGS. 2 and 3b.

The second generator includes an interphase circuit 114, an oscillator circuit 117, a converter-amplifier circuit 115, an on/off switch 119, a frequency divider circuit 116, a high-voltage plug 120 A, a high-voltage connection device comprising a plug 120 B, a cable 120 C, a connector 120 D and a capacitive application device 121. Their composition and functioning are exactly the same as the ones in the first high-voltage pulse generator.

Timer circuit 15 is shared by both generators, and their composition and functioning are exactly as shown in FIGS. 2 and 3b.

Interphase circuit 6 connects directly to lines A1 and A2 in the same way as the circuit shown in line 2, connecting line A1 with terminal B1 and line A2 with terminal B2 of interphase 8, On the contrary interphase circuit 114 interconnects with lines A1 and A2 via on/off switch 113 having two (2) poles and two (2) positions. In, the position of same phase “SF” connects line A1 with terminal B1 and line A2 with terminal B2 of interphase circuit 114. Under such conditions converter-amplifiers 10 and 115 via lines E1/E2 and E1′/E2′ respectively modulation wave in-phase F1 frequency, and therefore the positive and negative hemicycles will match in both amplifiers-converters, this making the polarity of high-voltage pulses matching in output H and in output H′. Switch 113 being in counter-phase “CF” (as drawn in diagram in FIG. 5) line A1 connects with terminal B2′ and line B′ of interphase circuit 114, under such conditions converters-amplifiers 10 and 115 will receive the tension wave of frequency F1 in counterphase, and therefore when converter-amplifier 10 will receive the positive hemicycle and through its output H will emit high-voltage negative pulses, converter-amplifier 115 will simultaneously receive the negative hemicycle of modulation wave and through its output H′ it will emit positive pulses; outputs H and H′ are emitting pulses having different polarities.

The low tension pulses signal enter converters-amplifiers 10 and 115 through line FP and FP′ via switches 11 and 119. The option of switches 11 and 119 in position A and A′ respectively, connects lines FP and FP′ simultaneously, and therefore both converters-amplifiers will receive low-tension 2×F1 frequency pulse wave thus the high-voltage pulses in both generators will be produced simultaneously; in the “in phase” functioning they will have the same polarity and in the “counterphase” functioning they will have opposite polarities. That is to say that in this case, because of being generated from same 2×F1 frequency pulses the damped high-voltage sinusoidal pulses will also be “in exact phase” o “in exact counterphase” depending on the position of switch 113 contact.

The option of switches 11 and 19 in position B and B′ respectively connects line FB of oscillator 9 with line FP of converter-amplifier 10 and enters in the latter the frequency F3 pulses, whereas terminal B′ of terminal 119 connects to common terminal of one of the two (2) poles of on/off switch 118, terminal “a” of the same pole connects to output FB of oscillator 9. In the other pole of switch 118 common terminal connects to input N′ of converter-amplifier 115 and on the other hand to terminal “c” of the same pole connects output N′ of divider circuit 116 and to terminal “d” output N of divider circuit 14. Switch 118 is an on/off switch with two (2) poles and two (2) positions: in position 1 it allows the independent functioning of both (2) high-voltage pulse generators because of connecting to converter-amplifier 115 with its own divider circuit 116 and oscillator 117, entering pulse frequency F3″ through line FP′ and intermittent frequency F2′through line N′ of converter-amplifier 115, which may be different from their respective frequencies.

On the contrary, switch 118 being in position “S”, both generators function in synchronism as both receive the same frequency F3 pulses and same intermittent frequency F2; this makes high-voltage pulses from outputs H and H′ to be in exact phase or in exact counterphase according to the position of switch 113.

The various combinations of the contact positions of switches 113, 118, 119 and 11 allow the embodiment of the invention with two (2) high-voltage pulse generators, functioning jointly insofar as possible.

The illustrated position of the capacitive application devices 13 and 121 in FIG. 5 correspond to the functioning “in counterphase” wherein the distances “d1” and “d2” indicate the separation between the active electrodes and the surface of the live system. Therefore the potential gradient between them will be as follows:
Ed₁=U₁₃/d₁ Ed₂=(−U₁₂₁)/d₂=U₁₂₁/d₂

wherein:

Ed₁=potential gradient of device 13.

Ed₂=potential gradient of device 121.

U₁₃=electrical gradient of the active electrode of device 13 for a determined instant

U₁₂₁=electrical potential of active electrode of device 121 for the same instant

Thus Ed₁ and Ed₂ have the same sense and originate a through electrical flow, this diminishing the dispersion and concentrating it on the area to be treated.

For the functioning with high-tension pulses in counter-phase, line “0” with potential “0” will always be situated near half distance separating both application devices; the live system to be treated is to placed in the middle of both (two) capacitive application devices.

For the apparatus functioning “in phase” the application devices 13 and 121 are to be placed side by side in a coplanar fashion.

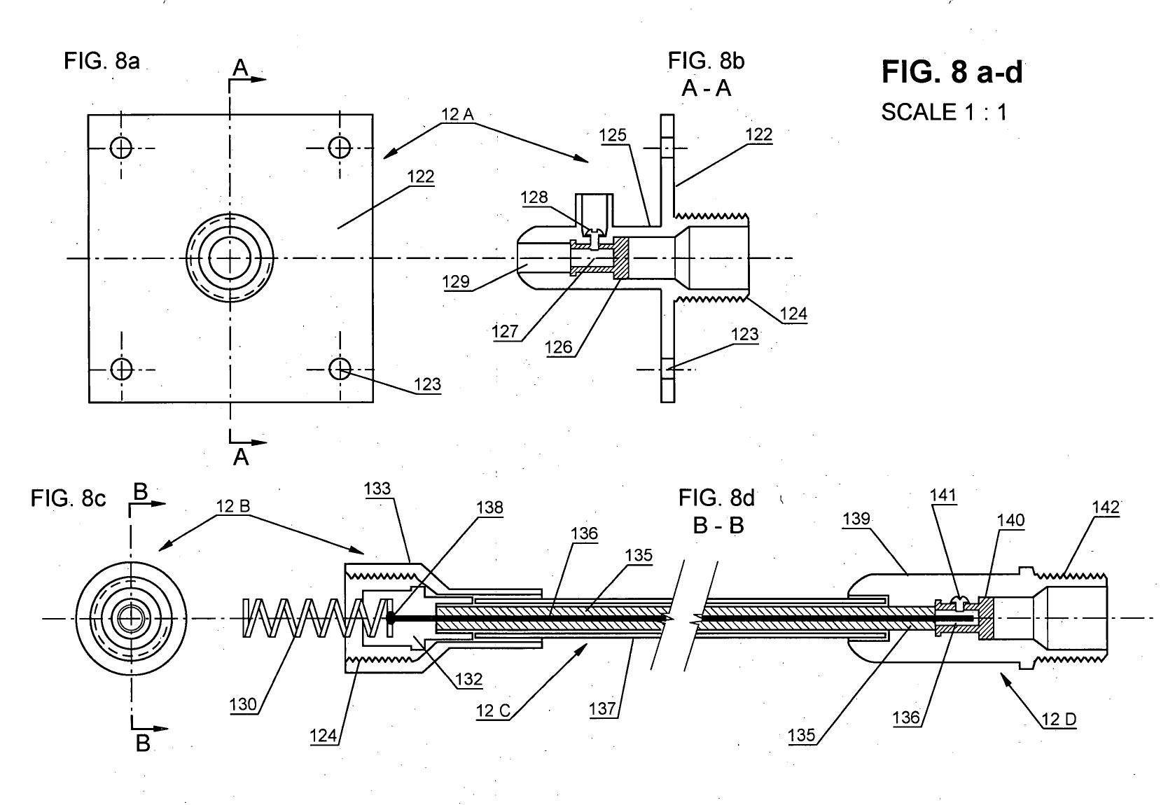

FIG. 8 shows a frontal view of high-tension socket 12A wherein frame 122 shows four (4) orifices 123 for affixing to the housing of the pulse generating apparatuses shown at FIGS. 2, 3b, 4 and 5.

FIG. 8b is a transversal section along section line A-A from FIG. 8a showing with more detail the structure of the high-voltage socket 12A wherein it is possible to appreciate frame 11, orifices 123, thread 124 for engaging plug 12B and the cylindrical body 125. All these parts are made of rigid high-impact PVC (polyvinyl chloride) or PP (Polypropylene), both injection molded with 99% purity and 1% of a colorant; the dielectric constant referred to vacuum shall be in the range of 3,1 to 3,3.

Cylindrical body 125 contains in its interior contact terminal 126 made of bronze; within the metallic terminal there is a cylindrical orifice 127 intended for receiving the connection copper cable which is pressed by screw 128.

Body 125 has a cylindrical orifice 129 which allow to introduce and cover the PVC isolation of the interconnection cable with terminal “H” of converter-amplifier 10; the tower above screw 128 confers a security space in case of non-intended contacts.

FIG. 8c is a frontal view of the interconnection device comprising the high-voltage plug 12B, cable 12C and connector 12D.

FIG. 8d is a transversal section along section line B-B from FIG. 8c showing with greater detail the design of the connection device, starting from compression spring 130 provided wit a electrolytic chroming surface covering, to be firmly mounted within the junction device 132 which on its turn is made of a plastics (PVC or PP); said device 132 on one side supports spring 130 and on the other side is solidarity joined to the high-tension cable formed by copper lead 136 and PVC isolation 135. Copper lead 136 and spring 130 are electrically joined by welding 138. This assembly is inserted into body 132 which can rotate and slide freely on union device 132 and cover 137 which is a transparent PVC hose serving as a mechanical protection for PVC isolation 135.

Body 133 is made of PVC or PP and contains in its interior a thread 134 which matches thread 124 from FIG. 8b. Spring 130 establishes electrical contact with terminal 126 which upon being introduced within socket 12a and staying there by means of body 133 and its thread which upon being engaged on thread 124 pushes union device 132 inwards thus compressing spring 130 with the required electrical contact pressure, and maintaining this connection firmly assured.

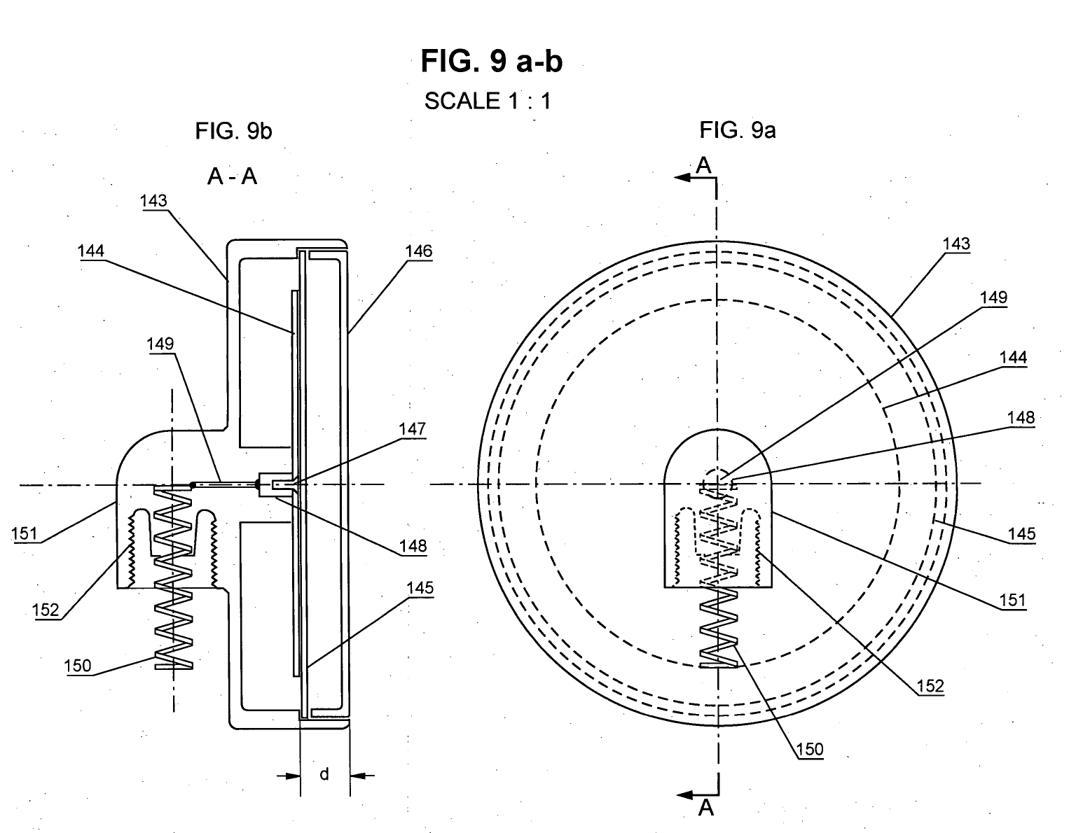

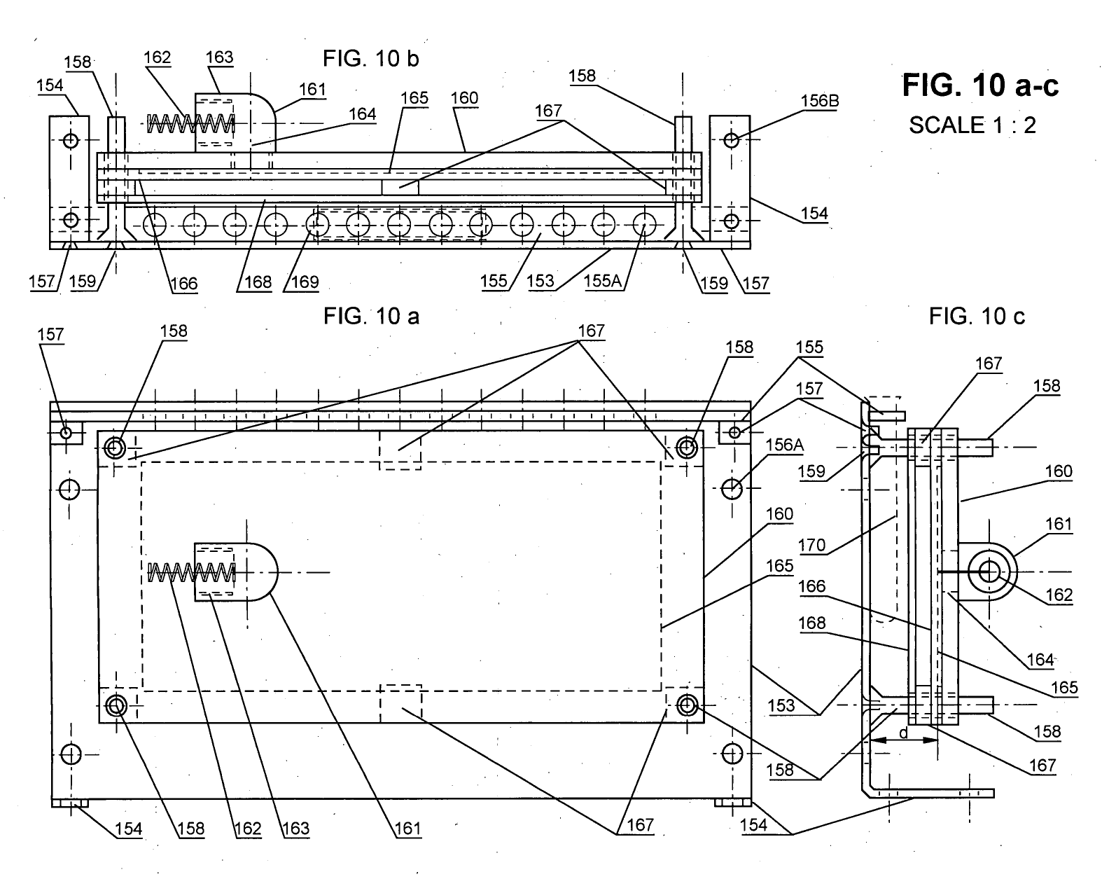

PVV isolation 135 is made of flexible PVC with the same dielectric features as above mentioned and its minimum thickness shall be 2,5 mm and its minimum external diameter of about 6 mm. Lead 136 will consist in a minimum of ten (10) electrolytic copper wires intended for electrical use 0,3 mm gauge each. At the end of connection cable 137 constituted by lead 136, isolation 135 and protection cover 137, connector 12D is situated constituted by a PVC or PP body 139 joined to isolation 135; lead 136 is introduced within contact terminal 140 having similar features as terminal 126 at FIG. 8, and is pressed by screw 141. The cable, terminal and screws, are totally covered by isolating body 139, the only ones left with an exit being the contact face of terminal 140, the free end of body 139 carries thread 142 similar to thread 124 of socket 12A of FIG. 8b. This threaded end of connector 12D is intended to connect to capacitive application device for biomedical treatments of FIG. 9a-b, as well for the capacitive application device for cultures of biological systems in test tubes, cfs. FIGS. 10a-c.

Body 133, junction device 132 and body 139 are made of rigid high impact PVC or POLYPROPYLENE, both injection molded with 99% purity and 1% of a colorant, their dielectric rigidity being in the range 40-50 KV/mm, and their dielectric rigidity referred to vacuum being 3,1-3,3.

FIG. 9a shows the capacitive application device for biomedical therapeutic treatments in human beings and higher animals, seen from the connection side, that is to say the side opposite the therapeutic application. In FIG. 9a, a transversal section along section line A-A from FIG. 9a is a more detailed view of the device structural design; both Figures show the top exterior cover of housing 143 made of PVC or POLYPROPYLENE, the electrically active electrode 144—which is a aluminum disc mounted on one side on an isolating rigid high-mechanical (high impact) resistance PVC, the latter being fitted within cover 143 thus forming with same a first air cavity under normal pressure. Disc 145 is affixed in this position by isolating lid 146, also made of rigid PVC or propylene, both forming another air cavity under normal pressure. Lid 146 is solidarity joined to cover 143, and is part of the capacitive application device resting on the surface of the body to be treated. Aluminum disc 144 is affixed by means of a galvanized milled head 147 with bronze insert 148 which is inserted in the plastic body of cover 143. Said insert is electrically joined by means of a cable 148 with spring 50, the latter having the same features as spring 130 of FIG. 8d. This spring 150 is fitted within head 151 of the house top, which has a thread 152 with the same features as thread 142 of connector 12D of FIG. 8d, this allows to threadedly engage the capacitive application device on connector 12D thus establishing an electrical contact between spring 150 of FIG. 9a-b and terminal 140 of FIG. 8d.

Housing 143 with its head 151 likewise to lid 146 from FIG. 9a-b are made of rigidly molded highly mechanically resistant PVC or Polypropylene with the same purity and dielectric features as in FIGS. 8a-b.

Distance “d” is the separation between metallic disc 144 or active electrode and external part of lid 146. When the capacitive application device is placed on the surface of the body to be treated, the patient being grounded by means of a reference electrode, in said separation “d” a potential gradient is established, Em, the values of which are comprised between 300 and 23,000 Volt/mm of maximum peak. Said potential gradient induces within the tissues electrical current density pulses with a value RMS having a power level such that no significative heating ensues. The biologically active penetration within the tissues comprises 4 to 15 cm depending on the tension value Upm of the high-voltage pulses of the activation signal, their minimum useful value being 3,000 Volt at peak.

En FIG. 10a the capacitive application device is shown for the treatment of cultures of biological systems in test tubes, seen from the connection side. FIG. 10b is un elevation view of the device shown at FIG. 10a; this view matches the horizontal utilization position for the treatment of cultures in a dry medium, wherein there is shown the placement of culture 169 between the support base 153 and the isolating separation plate 168. FIG. 10c is a lateral view of the device shown at FIG. 10a and is congruent with the vertical utilization position for the treatment of cultures in a liquid medium, the position of test tube 170 being illustrated between the support base 153 and the isolating separation plate 168. In said Figures it is possible to appreciate the support base 153 made of rustless steel plate with four (4) orifices 156A for affixing the device in horizontal; base 153 terminates in one of said sides 2 (two) lugs 154 which by means of orifices 156B allow the fixation of the device in vertical position. The support for the culture test tubes 155 is fixed by means of two (2) flanges in its ends with base 153 by means of two (2) milled head screws 156. Support 155 has a series of orifices 155A with a diameter sufficient for introducing and supporting culture test tubes 170 in vertical position.

The four (4) cylindrical supports 158 of millable plastics as for instance Delrim affixed to base 153 by means of milled head screws 159 are useful as supports and guide for the capacitive application device proper which by means of four (4) thru-holes may slide freely on the supports 158 thus selectively adjusting the separation distance from base 153.

Isolating plate 160 made of rigid high impact PVC forms the upper cover of device and the isolating plate 166, also made of rigid high impact PVC, forms the lower cover or the device and has a rectangular groove which receives an aluminum plate 165 constituting the active electrode of the application device. Isolating plates 160 and 166 are joined by means of an adhesive for PVC, o possibly by thermocompression.

Aluminum plate 165 is electrically joined via cable 164 with spring 162 by means of a contact pressure between the cable and the aluminum plate 165 and by means of welding between cable 164 and spring 162.

Spring 162 has the same features as spring 130 in FIG. 8d and is inserted in rigid molded plastic head 161, which is joined by means of a threaded hole 169A with isolating cover 160. Female thread 163 in head 161 allows the coupling with treading 142 of connector 12A shown in FIG. 8d and spring 162 establishes the electrical contact with terminal 140.

Isolating plate 168 made of rigid high impact PVC is joined by means of six (6) rigid PVC separators with lower cover 166. Said separators 167 establish an air separation distance between plate 168 and cover 166, this distance being necessary for avoiding electrical influences at the contact surface between the application device and the culture test tubes.

The dielectric characteristics of the isolating materials are the same as those indicated for above described cases.

Support base 153 connects directly to ground and distance “d” is the separation between this metallic base and active electrode 165, there being established between same a gradient potential Em as shown in FIG. 3b, the culture tube being immersed in this established electrical field y the biologically active values of which are comprised between 300 and 3,000 Volt/mm at maximum peak, this inducing in the biological cultures electrical current density pulses with a RMs value having a power level such that there is no significant heat generation. In this case also the useful minimum peak of the high-voltage pulses of the activation signal is 3,000 volt peak value.

Both for the treatment of human beings and higher animals as well for the treatment and development of biological system cultures, the electrical current pulses are the result of the movement of ions in the interior and exterior of cells, said movement being caused by the impulsive attractive and repulsive forces due to the potential gradient action of the high-voltage on the electrical charges of the ions, this producing a physical agitation such that chemical reactions accelerate (the system acquires kinetic energy) and interactuates al the level of cellular membrane in the ionic passage between the cells and the intercellular fluids, this producing an increase in the enzymatic activity, and thus influencing the cellular metabolic reactions, without causing any significant heating on the live system treated.

The apparatus has been described for use in the treatment of live systems both for promoting the healing of soft and osseous tissues in biomedical applications on human beings and higher animals and for accelerating the growing and development of cellular cultures, bacterial cultures, etc., through the electroionic cellular agitation achieved with the means here-above described.

The application field of the apparatus is not restricted to promoting the cellular healing; it can also be used for influencing other metabolic processes including the action on substances, drugs, pharmaceuticals, etc., having an ionic nature upon solubilizing in blood or in other bodily fluids.

Having thus described the apparatus with regard to some specific embodiments of same, it is to be understood that their description is not intended to limit the scope of the invention, as further modifications suggested themselves to a person with the usual skill in the art, the aim being to encompass such modifications within the scope of adjoined claims.

1. Electroionic cellular agitation apparatus for treating a live system by influencing the metabolic parameters for the growing of the live system, the apparatus comprising:
means for applying electric flow pulses to the live system to generate within the ions located inside the cells of the live systems, short duration impulsive forces which cause an ionic agitation inside said cells in order to influence the growth metabolic parameters;
said electric pulses having a predetermined characteristic and a power level such that the RMS value of the density of the capacitively induced electrical current is sufficient for increasing the enzymatic efficiency and influencing the growth metabolic parameters, but is also provided with a power level such that no significant heating is effected on the live system.

2. Apparatus according to claim 1, wherein said application means comprise at least one generator for generating high-voltage pulses and up to a maximum of two of such generators; and the means for the rhythmical turning-on which connect the generator control for an intermittent operation, said periodic intervals defining the treatment time and the global duration of same.

3. Apparatus according to claim 2, wherein said generator of high-voltage pulses comprises an oscillator outputting low voltage low-frequency pulses typically below 1,000 pulses/sec; said oscillator being connected to a converter-amplifier which modifies and increases the tension of said pulses; and a low-frequency oscillator-ondulator typically in the 5-200 Hz range feeding said converter-amplifier by modulating the amplitude of said high-voltage pulses and by alternating the polarity of same; in one hemicycle of said modulation wave the pulses are positive, and in the other hemicycle of said modulation wave the pulses are negative; thus generating an activation signal consisting in high-voltage pulses, applied to a capacitive application device placed near the area to be treated and generating an electrical flow which causes a biophysical stimulation on said area to be treated.

4. Apparatus according to claim 2, wherein said means for the rhythmical turning-on define a sequence of periodical cycles and allow the operation of said high-voltage pulse generator for a predetermined portion of each periodical cycle and define the global duration of the treatment time.

5. Apparatus according to claim 3, further comprising means for selecting the duration of the emission times and pause times of said periodical cycle and the global treatment time; and the display means for showing the values for said times for which the apparatus is being operated.

6. Apparatus according to claim 3 wherein the means for controlling said low-voltage low-frequency generator comprise the means for manually selecting the value for said frequency and the means for automatically varying the value for said frequency.

7. Apparatus according to claim 6 comprising display means for showing the value for said pulse frequency for which the apparatus is being operated.

8. Apparatus according to claim 3 further comprising means for manually and automatically selecting the polarity of said high-voltage pulses, and means for displaying and indicating said polarity for which the apparatus is being operated.

9. Apparatus according to claim 3 further comprising means for selecting the peak tension values for said high-voltage pulses; and means for displaying and indicating said peak tension value, for which the apparatus is being operated.

10. Apparatus according to claim 3 further comprising detection means for checking the correct operation of the apparatus.

11. Apparatus according to claim 3, further comprising means for selecting the values for said amplitude modulation frequency and means for displaying the indication for said modulation frequency for which the apparatus is being operated.

12. Apparatus according to claim 2 further comprising means for selecting the simultaneous operation of said two high-voltage pulse generators, such that both work in-phase, wherein each of said capacitive application devices is placed on a same plane on the area to be treated; or in counter-phase wherein each of said capacitive application devices is placed in front of the system to be treated, the latter being placed in the middle of both devices.

13. Apparatus according to claim 12 further comprising means for displaying the indication of the simultaneous functioning mode of said high-voltage pulse generators, for which the apparatus is being operated.

14. Apparatus according to claim 3, said activation signal of high-voltage pulses being applied to at least one capacitive application device, the latter being placed near the area to be treated, for the biophysical stimulation of the live system, wherein said activation comprises:
a high-voltage pulse train the amplitude of which is modulated by a treatment wave having a relatively low frequency typically between approximately 5 and 200 Hz, said modulation wave further determining the electric polarity of said high-voltage pulses; said pulses being positive in one hemicycle of said modulation cycle and negative in the other hemicycle and so on repeatedly;
each of said high-voltage pulses having a strongly damped sinusoidal wave wherein the first peak determines the electrical neat polarity of the pulse; the second peak with a polarity opposed to the first one typically attains no more than approximately 60% of the voltage value of the first peak; the third peak has same polarity as the first one and typically attains no more than approximately 25% of the fist peak value, and after said third peak the electrical polarity inverts again thus forming a slow varying land with a tension less than 12% of the first peak value and with a duration equal to 1,2 times the total duration time of the damped pulsation, till the onset of next pulse,
each of said pulses having a wave frequency typically below approximately 3,000 Hz,
said high-voltage pulse train, amplitude-modulated by a relatively low-frequency wave, attains typically a peak maximum maximorum value for the first pulse peak of no less than approximately 3,000 volts as measured between the output of said high-tension pulses and ground.

15. Apparatus according to claim 3, said capacitive application device being placed near the live system to be treated, for accelerating the development and growth of said live system, the live systems including bacterial and fungal cultures, soft and osseous live tissues; as well for shortening the production time of the subproducts possibly issuing from said live systems, said capacitive application device comprising:
a metallic plate electrically fully isolated from the system to be treated and the surrounding medium, said metallic plate being connected to the output of said high-voltage generator thus conforming the fully isolated electrode of the capacitive application device and a metallic plate connected to earth and the mass the high-voltage generator, there being a separation between said plate and said completely isolated electrode which allows to place between both the live system to be treated; and
wherein upon said activation signal being applied for the high-voltage pulses to said active electrode, it develops an electrical field between electrode and the grounded plate, with the same pulse form and frequency as said activation signal for the high-voltage pulses which originated it; and
wherein said electric field pulses attain maximum maximorum peak values typically not above approximately 3,000 vol/mm and not below approximately 300 vol/mm and wherein said pulses from the electrical field develop an electrical current flow capacitively induced in the live system, the RMS value of the electrical current density has a power level such that no significant heat ensues on the system treated.

16. Apparatus according to claim 1, said capacitive application device being placed near the live system to be treated for the biomedical stimulation, treating a variety of ailments, achieving an anticipated healing and the reduction of rehabilitation times, in human beings and higher animals, said capacitive application device comprising:
a metallic plate electrically fully isolated from the system to be treated and the surrounding medium, said metallic plate being connected to the output of said high-voltage generator thus conforming the fully isolated electrode of the capacitive application device and the mass of the high-voltage generator being connected to mass and the patient being referred to earth by direct contact or through a capacitive contact; and
wherein upon said activation signal being applied for the high-voltage pulses to said active electrode, its develops an electrical field between electrode and the patient with the same pulse form and frequency as said activation signal for the high-voltage pulses which originated it; and wherein said electric field pulses attain maximum maximorum peak values typically not above approximately 3,000 vol/mm and not below approximately 300 vol/mm; and
wherein said pulses from the electrical field develop an electrical current flow capacitively induced in the live system, the RMS value of the electrical current density has a power level such that no significant heat ensues on the area being treated nor in any other part of patient's body.

17. A method for influencing the growth metabolic characteristics of live systems, for accelerating the development and growth of said system, including bacterial and fungal cultures, soft and osseous live tissues, as well for shortening the production time for the subproducts liable to issue from the metabolic activity of said live systems, said method comprising following steps:
using at least one capacitive application device having its active pole electrically completely isolated from the live system and surrounding medium.
using an activation signal for high-voltage pulses having a peak value typically not below 7,000 volt, applied to said totally isolated active pole, and placing the live system between said capacitive application device and a metallic plate referred to earth, this generating electrical field pulses with the same waveform and frequency of the activation signal and values for the maximum maximorum peak not above 3,000 volt/mm and not below approximately 300 volt/mm, and
wherein said pulses from the electrical field penetrate the live system, thus causing short duration impulsive attractive and repulsive forces on the ions within the cells and the intercellular fluids, this originating a ionic agitation, increasing the enzymatic efficiency, the ionic interchange and influencing the growth metabolic parameters; and
wherein said pulses from the electrical field develop an electrical current flow capacitively induced in the live system, the RMS value of the electrical current density has a power level such that no significant heat ensues on said live system.

18. A therapeutic method for human beings and higher animals for treatment of a variety of ailments, attaining an anticipated healing and reduction in rehabilitation times, comprising following steps:
using at least one capacitive application device having its active pole electrically completely isolated from the patient to be treated and surrounding medium; and using an activation signal for high-voltage pulses having a peak value typically not below 3,000 volt, applied to said totally isolated active pole;
said capacitive application device being placed near the area to be treated and the patient being electrically referred to earth, that is, in a capacitive way or directly connected by a conductive electrode; and
thus generating electrical field pulses with the same waveform and frequency of the activation signal and values for the maximum maximorumn peak not above 3,000 volt/mm and not below 300 volt/mm, and
wherein said electrical field pulses penetrate the tissue in the area to be treated;
this causing short duration electrical attractive and repulsive impulsion forces on the ions within the cells and intercellular fluids, this originating
the ionic agitation of said ions, increasing the enzymatic efficiency, the ionic exchange and influencing the growth metabolic parameters; and
wherein said agitation of the ionic ions constitute the capacitively induced electric current on the cellular tissues in the area to be treated, the RMS value of the electrical current density has a power level such that no significant heat ensues on said area to be treated nor in any other part of patient's body.

19. Method according to claim 18, at least one second capacitive application device being placed facing directly the first one and the part of patient's body to be treated being placed between both capacitive application devices, and by applying to said second capacitive application device an activation signal for high-voltage with directly opposed signs, and subsequently generating on either sides of the part to be treated electrical fields with equal direction and sense, in such a way to add both electrical field intensities and attaining a greater penetration and lesser dispersion in the part to be treated of patient's body.
